(19) United States
(12) Patent Application Publication
(10) Pub. No.: US 2022/0000751 A1
(43) Pub. Date: Jan. 6, 2022

(54) METHOD FOR PREPARING AND USING HAIR TREATMENT AGENTS WITH ORGANIC C1-C6-ALKOXY-SILANES

(71) Applicant: Henkel AG & Co. KGaA, Duesseldorf (DE)

(72) Inventors: Torsten LECHNER, Langenfeld (DE); Christoph LOHR, Mettmann (DE); Andreas WALTER, Ratingen (DE); Claus-Peter THIESSIES, Duesseldorf (DE); Juergen SCHOEPGENS, Schwalmtal (DE)

(73) Assignee: Henkel AG & Co. KGaA, Duesseldorf (DE)

(21) Appl. No.: 17/436,036

(22) PCT Filed: Jan. 24, 2020
(86) PCT No.: PCT/EP2020/051823

§ 371 Date: Sep. 2, 2021

(51) Int. Cl. A61K 8/58; A61Q 5/10

(52) U.S. Cl. CPC A61K 8/585; A61K 2800/88; A61Q 5/10

(57) ABSTRACT

The subject of the present application is a process for the preparation of an agent for the treatment of keratinous material, in particular human hair, comprising the following steps:
(1) Mixing one or more organic C₁-C₆ alkoxy silanes with water,
(2) partial or complete removal from the reaction mixture of the C₁-C₆ alcohols released by the reaction in step (1),
(3) optionally addition of one or more cosmetic ingredients, and
(4) Filling of the preparation into a packaging unit,
exemplified in that the total content of C₁-C₆ alcohols in the preparation obtained in step (4)—based on the total weight of the preparation—is below about 10.0% by weight.

### CROSS-REFERENCE TO RELATED APPLICATIONS

This application is a U.S. National-Stage entry under 35 U.S.C. § 371 based on International Application No. PCT/EP2020/051823, filed Jan. 24, 2020, which was published under PCT Article 21(2) and which claims priority to German Application No. 10 2019 203 081.5, filed Mar. 6, 2019, which are all hereby incorporated in their entirety by reference.

### TECHNICAL FIELD

The present application is in the field of cosmetics and concerns a process for the preparation of hair treatment compositions, and a kit of parts for hair treatment.

### BACKGROUND

In the process as contemplated herein, one or more organic C₁-C₆ alkoxy silanes are reacted with water, and the C₁-C₆ alcohols released in this reaction are removed as completely as possible from the reaction mixture. As further steps, the method as contemplated herein optionally comprises the addition of one or more cosmetic ingredients to this preparation and the filling of the preparation into a packaging unit.

A second article is an agent for treating keratinous material, comprising a preparation in a packaging unit prepared according to the methods.

A third object of the present disclosure is a multi-component packaging unit (kit-of-parts) for coloring keratinous material, which comprises, separately packaged in two packaging units, the cosmetic preparations (A) and (B), the preparation (A) being a preparation of the first object of the present disclosure and the preparation (B) containing at least one coloring compound.

The change in shape and color of keratin fibers, especially hair, is an important area of modem cosmetics. To change the hair color, the expert knows various coloring systems depending on coloring requirements. Oxidation dyes are usually used for permanent, intensive dyeing's with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the colorant into the hair fiber. Compared to oxidative hair dyeing, the dyeing's obtained with direct dyes have a shorter shelf life and quicker wash ability. Dyeing with direct dyes usually remain on the hair for a period of between about 5 and about 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. Therefore, they can usually be removed again without residue by a few washes with detergents containing surfactants. Various products of this type are available on the market under the name hair mascara.

If the user wants particularly long-lasting dyeing's, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair.

EP 2168633 B1 deals with the task of producing long-lasting hair colorations using pigments. The paper teaches that when a combination of pigment, organic silicon compound, hydrophobic polymer and a solvent is used on hair, it is possible to produce colorations that are particularly resistant to shampooing.

The organic silicon compounds used in EP 2168633 B1 are reactive compounds from the class of alkoxy silanes. These alkoxy silanes hydrolyze at high rates in the presence of water and form hydrolysis products and/or condensation products, depending on the amounts of alkoxy silane and water used in each case. The influence of the amount of water used in this reaction on the properties of the hydrolysis or condensation product are described, for example, in WO 2013068979 A2.

When these hydrolysis or condensation products are applied to keratinous material, a film or coating is formed on the keratinous material, which completely envelops the keratinous material and, in this way, strongly influences the properties of the keratinous material. Possible areas of application include permanent styling or permanent shape modification of keratin fibers. In this process, the keratin fibers are mechanically shaped into the desired form and then fixed in this form by forming the coating described above. Another particularly suitable application is the coloring of keratin material; in this application, the coating or film is produced in the presence of a coloring compound, for example a pigment. The film colored by the pigment remains on the keratin material or keratin fibers and results in surprisingly wash-resistant colorations.

The great advantage of the alkoxy silane-based dyeing principle is that the high reactivity of this class of compounds enables extremely fast coating. This means that extremely good coloring results can be achieved after short application periods of just a few minutes. In addition to these advantages, however, the high reactivity of alkoxy silanes also has some disadvantages. Thus, even minor changes in production and application conditions, such as changes in humidity and/or temperature, can lead to sharp fluctuations in product performance. Most importantly, the work leading to this disclosure has shown that the alkoxy silanes are extremely sensitive to the conditions encountered in the manufacture of the keratin treatment agents.

Analytical studies have shown that complex hydrolysis and condensation reactions take place during the preparation of various silane mixtures and blends, leading to oligomeric products of different molecular size depending on the reaction conditions selected. In this context, it has been found that the molecular weight of these silane oligomers can have a major influence on the subsequent product properties. If wrong conditions are selected during production, this can lead to the formation of silane condensates that are too large or too small, which negatively affects the subsequent product performance, especially the subsequent dyeing capacity on the keratin material.

### BRIEF SUMMARY

Treatment agents for keratinous materials and methods of preparing the same are provided. In an exemplary embodiment, a method for preparing an agent for treating keratinous material includes mixing one or more C₁-C₆ alkoxy silanes with water to produce a reaction mixture. C₁-C₆ alcohols are produced in the reaction mixture by a reaction between the one or more C₁-C₆ alkoxy silanes and water, and the C₁-C₆ alcohols are removed. One or more cosmetic ingredients are optionally added to the reaction mixture, and a preparation produced by the reaction mixture is filled into a packaging unit. The total content of C₁-C₆ alcohols in the preparation is below about 10% by weight, based on a total weight of the preparation.

An agent for treating keratinous material is provided in another embodiment. The agent includes a preparation produced by mixing a C₁-C₆ alkoxy silane with water to produce a reaction mixture that generates a C₁-C₆ alcohol. The C₁-C₆ alcohol is removed from the reaction mixture, and a cosmetic agent is optionally added to the reaction mixture. The preparation is produced from the reaction mixture with a C₁-C₆ alcohol content of less than about 10 weight percent, based on a total weight of the preparation. The preparation is filled into a packaging unit.

A multicomponent packaging unit for dying keratinous material is provided in another embodiment. The multicomponent packaging unit includes a cosmetic preparation (A) in a first packaging unit and a cosmetic preparation (B) in a second packaging unit. The cosmetic preparation (A) is produced by mixing a C₁-C₆ alkoxy silane with water to produce a reaction mixture that generates a C₁-C₆ alcohol. The C₁-C₆ alcohol is removed from the reaction mixture, and a cosmetic agent is optionally added to the reaction mixture. The preparation (A) is produced from the reaction mixture with a C₁-C₆ alcohol content of less than about 10 weight percent, based on a total weight of the preparation (A). The preparation (A) is filled into the first packaging unit. The second packaging unit includes a colorant selected from pigments, direct dyes, and/or oxidation dye precursors.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It was the task of the present application to find an optimized process to produce keratin treatment agents. The alkoxy silanes used in this process were to be prepared in a targeted manner so that the optimum application properties could be achieved in a subsequent application. In particular, the agents prepared by this method should have improved dyeing performance, i.e., when used in a dyeing process, dyeing's with higher color intensity and improved fastness properties, especially improved wash fastness and improved rub fastness, should be obtained.

Surprisingly, it has now been found that the task can be excellently solved if a production process is selected in which a selective hydrolysis of organic C₁-C₆ alkoxy silanes is carried out and the alcohols released in this reaction are removed as completely as possible. If necessary, further cosmetic ingredients can be added to the preparations prepared in this way. Then the filling into a packaging unit takes place.

A first object of the present disclosure is a method for preparing an agent for treating keratinous material, in particular human hair, comprising the following steps:
(1) Mixing one or more organic C₁-C₆ alkoxy silanes with water,
(2) partial or complete removal from the reaction mixture of the C₁-C₆ alcohols released by the reaction in step (1),
(3) optionally addition of one or more cosmetic ingredients, and
(4) Filling of the preparation into a packaging unit, exemplified in that the total content of C₁-C₆ alcohols in the preparation obtained in step (4)—based on the total weight of the preparation—is below about 10.0% by weight.

It has been shown that hair treatment compositions prepared by this process as contemplated herein, when used in a dyeing process, resulted in very intense and uniform colorations with particularly good coverage, rub fastness and wash fastness.

### Agent for the Treatment of Keratinous Material

Keratinous material includes hair, skin, and nails (such as fingernails and/or toenails). Wool, furs, and feathers also fall under the definition of keratinous material.

Preferably, keratinous material is understood to be human hair, human skin, and human nails, especially fingernails and toenails. Keratinous material is understood to be human hair.

Agents for treating keratinous material are understood to mean, for example, techniques for coloring the keratinous material, techniques for reshaping or shaping keratinous material, in particular keratinous fibers, or also techniques for conditioning or caring for the keratinous material. The agents prepared by the process of the present disclosure are particularly suitable for coloring keratinous material, in particular keratinous fibers, which are preferably human hair.

The term “coloring agent” is used in the context of the present disclosure to refer to a coloring of the keratin material, of the hair, caused using coloring compounds, such as thermochromic and photochromic dyes, pigments, mica, direct dyes and/or oxidation dyes. In this staining process, the colorant compounds are deposited in a particularly homogeneous and smooth film on the surface of the keratin material or diffuse into the keratin fiber. The film is formed in situ by oligomerization or condensation of the organic silicon compound(s), with the colorant compound(s) interacting with or being incorporated into this film or coating.

### Mixing C₁-C₆ Alkoxy Silane(s) with Water

Step (1) of the process as contemplated herein involves the reaction or also reaction of one or more organic C₁-C₆ alkoxy silanes with water. To initiate this reaction, the C₁-C₆ alkoxy silane(s) are mixed with water.

In other words, the first object of the present disclosure is a method for preparing an agent for treating keratinous material, in particular human hair, comprising the following steps:
(1) Reaction of one or more organic C₁-C₆ alkoxy silanes with water,
(2) partial or complete removal from the reaction mixture of the C₁-C₆ alcohols released by the reaction in step (1),
(3) optionally addition of one or more cosmetic ingredients, and
(4) Filling of the preparation into a packaging unit, exemplified in that the total content of C₁-C₆ alcohols in the preparation obtained in step (4)—based on the total weight of the preparation—is below about 10.0% by weight.

The organic C₁-C₆ alkoxy silane(s) are organic, non-polymeric silicon compounds, preferably selected from the group of silanes containing one, two or three silicon atoms.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds of the present disclosure are preferably compounds containing one to three silicon atoms. Organic silicon compounds preferably contain one or two silicon atoms.

According to IUPAC rules, the term silane chemical compounds is based on a silicon skeleton and hydrogen. In organic silanes, the hydrogen atoms are completely or partially replaced by organic groups such as (substituted) alkyl groups and/or alkoxy groups.

An exemplary feature of the C₁-C₆ alkoxy silanes of the present disclosure is that at least one C₁-C₆ alkoxy group is directly bonded to a silicon atom. The C₁-C₆ alkoxy silanes as contemplated herein thus comprise at least one structural unit R′R″R′″Si—O—(C₁-C₆ alkyl) where the radicals R′, R″ and R′″ stand for the three-remaining bond valencies of the silicon atom.

The C₁-C₆ alkoxy group or groups bonded to the silicon atom are very reactive and are hydrolyzed at high rates in the presence of water, the reaction rate depending, among other things, on the number of hydrolysable groups per molecule. If the hydrolysable C₁-C₆ alkoxy group is an ethoxy group, the organic silicon compound preferably contains a structural unit R′R″R′″Si—O-CH2-CH3. The R′, R″ and R′″ residues again represent the three remaining free valences of the silicon atom.

In a very particularly preferred embodiment, a process as contemplated herein is exemplified in that in step (1), one or more organic C₁-C₆ alkoxy silanes selected from silanes having one, two or three silicon atoms are reacted with water, the organic silicon compound further comprising one or more basic chemical functions.

This basic group can be, for example, an amino group, an alkylamino group or a dialkylamino group, which is preferably connected to a silicon atom via a linker. Preferably, the basic group is an amino group, a C₁-C₆ alkylamino group or a di(C₁-C₆)alkylamino group.

A very particularly preferred method as contemplated herein is exemplified by the

(1) Mixing one or more organic C₁-C₆ alkoxy silanes with water, wherein the organic C₁-C₆ alkoxy silanes are selected from the group of silanes having one, two or three silicon atoms, and wherein the C₁-C₆ alkoxy silanes further comprise one or more basic chemical functions.

Particularly good results were obtained when C₁-C₆ alkoxy silanes of formula (I) and/or (II) were used in the process as contemplated herein.

In another very particularly preferred embodiment, a method as contemplated herein is exemplified by the
(1) Mixing one or more organic C₁-C₆ alkoxy silanes of formula (I) and/or (II) with water,

R₁R₂N-L-Si(OR₃)ₐ(R₄)_{b} (I)

where
R₁, R₂ independently represent a hydrogen atom or a C₁-C₆ alkyl group,
L is a linear or branched divalent C₁-C₂₀ alkylene group,
R3, R4 independently of one another represent a C₁-C₆ alkyl group,
a, stands for an integer from 1 to 3, and
b stands for the integer 3-a, and

(R₅O)_{c}(R₆)_{d}Si-(A)ₑ—[NR₇-(A′)]_{f}-[O-(A″)]_{g}-[NR₈-(A′″)]h-Si(R₆′)_{d′}(OR₅′)_{c′} (II),

where
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III),

(A″″)-Si(R₆″)_{d}″(OR₅″)_{c}″ (III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g, and h is different from 0.

The substituents R₁, R₂, R₃, R₄, R₅, R₅′, R₅″, R₆, R₆′, R₆″, R₇, R₈, L, A, A′, A″, A′″ and A″″ in the compounds of formula (I) and (II) are explained below as examples:

Examples of a C₁-C₆ alkyl group are the groups methyl, ethyl, propyl, isopropyl, n-butyl, s-butyl, and t-butyl, n-pentyl and n-hexyl. Propyl, ethyl, and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂), the ethylene group (—CH₂—CH₂—), the propylene group (—CH₂—CH₂—CH₂—) and the butylene group (—CH₂—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. Very preferably, R₁ and R₂ both represent a hydrogen atom.
In the middle part of the organic silicon compound is the structural unit or the linker -L-which stands for a linear or branched, divalent C₁-C₂₀ alkylene group. The divalent C₁-C₂₀ alkylene group may alternatively be referred to as a divalent or divalent C₁-C₂₀ alkylene group, by which is meant that each—L grouping may form—two bonds.
Preferably -L- stands for a linear, divalent C₁-C₂M alkylene group. Further preferably -L-stands for a linear divalent C₁-C₆ alkylene group. Particularly preferred -L stands for a methylene group (CH₂-), an ethylene group (—CH₂—CH₂—), propylene group (—CH₂—CH₂—CH₂—) or butylene (—CH₂—CH₂—CH₂—CH₂—). Preferably L stands for a propylene group (—CH₂—CH₂—CH₂—)

The organic silicon compounds of formula (I)

R₁R₂N-L-Si(OR₃)ₐ(R₄)_{b} (I),

each carries the silicon-containing group —Si(OR₃)ₐ(R₄)_{b} at one end.
In the terminal structural unit —Si(OR₃)ₐ(R₄)_{b}, R₃ and R₄ independently represent a C₁-C₆ alkyl group, and particularly preferably R₃ and R₄ independently represent a methyl group or an ethyl group.
Here a stands for an integer from 1 to 3, and b stands for the integer 3-a. If a stands for the number 3, then b is equal to 0. If a stands for the number 2, then b is equal to 1. If a stands for the number 1, then b is equal to 2.

Keratin treatment agents with particularly good properties could be prepared if in step (1) at least one organic C₁-C₆ alkoxy silane of formula (I) was mixed with water or reacted, in which the radicals R₃, R₄ independently of one another represent a methyl group or an ethyl group.

Furthermore, dyeing's with the best wash fastnesses could be obtained when at least one organic C₁-C₆ alkoxy silane of formula (I) was reacted with water in step (1), in which the radical a represents the number 3. In this case the remainder b stands for the number 0.

In another preferred embodiment, a process as contemplated herein is exemplified in that in step (1) one or more organic C₁-C₆ alkoxy silanes of formula (I) are mixed with water,

where
R₃, R₄ independently of one another represent a methyl group or an ethyl group and
a stands for the number 3 and
b stands for the number 0.

In a further preferred embodiment, a process as contemplated herein is exemplified in that in step (1) one or more organic C₁-C₆ alkoxy silanes of formula (I) and/or (II) are mixed or reacted with water,

R₁R₂N-L-Si(OR₃)ₐ(R₄)_{b} (I),

where
R₁, R₂ both represent a hydrogen atom, and
L represents a linear, divalent C₁-C₆-alkylene group, preferably a propylene group (—CH₂—CH₂—CH₂—) or an ethylene group (—CH₂—CH₂—),
R₃ represents an ethyl group or a methyl group,
R₄ represents a methyl group or an ethyl group,
a stands for the number 3 and
b stands for the number 0.

Organic silicon compounds of the formula (I) which are particularly suitable for solving the problem as contemplated herein are

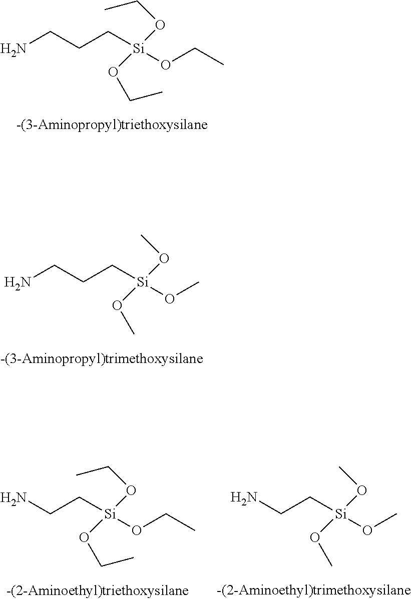

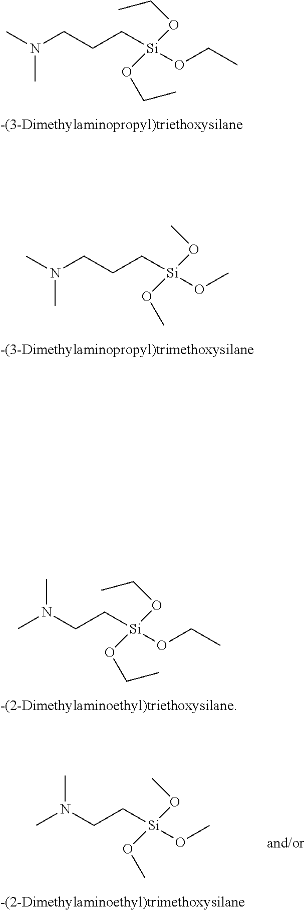

In a further preferred embodiment, a process as contemplated herein is exemplified in that in step (1) one or more organic C₁-C₆ alkoxy silanes is selected from the group of (3-Aminopropyl)triethoxysilane

### (3-Aminopropyl)trimethoxysilane

### -1-(3-Aminopropyl)silantriol

### (2-Aminoethyl)triethoxysilane

### (2-Aminoethyl)trimethoxysilane

### -1-(2-Aminoethyl)silantriol

### (3-Dimethylaminopropyl)triethoxysilane

### (3-Dimethylaminopropyl)trimethoxysilane

### -1-(3-Dimethylaminopropyl)silantriol

### (2-Dimethylaminoethyl)triethoxysilane.

(2-Dimethylaminoethyl)trimethoxysilane and/or

### -1-(2-Dimethylaminoethyl)silantriol

mixed with water or made to react.

The organic silicon compound of formula (I) is commercially available. (3-aminopropyl)trimethoxysilane, for example, can be purchased from Sigma-Aldrich®. Also (3-aminopropyl)triethoxysilane is commercially available from Sigma-Aldrich®.

In a further embodiment of the process as contemplated herein, one or more organic C₁-C₆ alkoxy silanes of formula (II) may also be mixed with water or reacted in step (1),

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II).

The organosilicon compounds of formula (II) as contemplated herein each carry the silicon-containing groups (R₅O)_{c}(R₆)_{d}Si— and —Si(R₆′)_{d′}(OR₅′)_{c} at both ends.

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g, and h can independently of one another stand for the number 0 or 1, with the proviso that at least one of the radicals e, f, g, and h is different from 0. In other words, an organic silicon compound of formula (II) as contemplated herein contains at least one grouping from the group of -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d′}(OR₅′)_{c}′, the residues R5, R5′, R5″ independently represent a C₁-C₆ alkyl group. The radicals R6, R6′ and R6″ independently represent a C₁-C₆ alkyl group.

Here c stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

Dyeing's with the best wash fastness values could be obtained if the residues c and c′ both stand for the number 3. In this case d and d′ both stand for the number 0.

In a further preferred embodiment, a process as contemplated herein is exemplified in that in step (1) one or more organic C₁-C₆ alkoxy silanes of formula (II) are mixed or reacted with water,

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
R5 and R5′ independently represent a methyl group or an ethyl group,
c and c′ both stand for the number 3 and
d and d′ both stand for the number 0.

If c and c′ are both the number 3 and d and d′ are both the number 0, the organic silicon compound of the present disclosure corresponds to formula (IIa)

(R₅O)₃Si-(A)ₑ-[NR₇-(A′)]_{f}-[O-(A″)]_{g}-[NR₈-(A′″)]ₕ—Si(OR₅′)₃ (IIa).

The radicals e, f, g, and h can independently stand for the number 0 or 1, whereby at least one radical from e, f, g, and h is different from zero. The abbreviations e, f, g, and h thus define which of the groupings -(A)ₑ- and —[NR₇-(A′)]f- and —[O-(A″)]_{g}- and —[NR₈-(A′″)]ₕ- are in the middle part of the organic silicon compound of formula (II).

In this context, the presence of certain groupings has proven to be particularly advantageous in terms of achieving washfast dyeing results. Particularly good results could be obtained if at least two of the residues e, f, g, and h stand for the number 1. Especially preferred e and f both stand for the number 1. Furthermore, g and h both stand for the number 0.

If e and f both stand for the number 1 and g and h both stand for the number 0, the organic silicon compound as contemplated herein corresponds to formula (IIb)

(R₅O)_{c}(R₆)_{d}Si-(A)-[NR₇-(A′)]-Si(R₆′)_{d′}(OR₅′)_{c′} (IIb).

The radicals A, A′, A″, A′″and A″″ independently represent a linear or branched divalent C₁-C₂₀ alkylene group. Preferably the radicals A, A′, A″, A′″ and A″″ independently of one another represent a linear, divalent C₁-C₂₀ alkylene group. Further preferably the radicals A, A′, A″, A′″ and A″″ independently represent a linear divalent C₁-C₆ alkylene group.

The divalent C₁-C₂₀ alkylene group may alternatively be referred to as a divalent or divalent C₁-C₂₀ alkylene group, by which is meant that each grouping A, A′, A″, A′″ and A″″ may form two bonds.

In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the radicals A, A′, A″, A′″ and A″″ stand for a propylene group (—CH₂—CH₂—CH₂—).

If the radical f represents the number 1, then the organic silicon compound of formula (II) as contemplated herein contains a structural grouping —[NR₇-(A′)]-.

If the radical h represents the number 1, then the organic silicon compound of formula (II) as contemplated herein contains a structural grouping —[NR₈-(A′″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

(A″″)-Si(R₆″)_{d}″((OR₅″)_{c}″ (III).

Very preferably the radicals R7 and R8 independently of one another represent a hydrogen atom, a methyl group, a 2-hydroxyethyl group, a 2-alkenyl group, a 2-aminoethyl group or a grouping of the formula (III).

If the radical f represents the number 1 and the radical h represents the number 0, the organic silicon compound as contemplated herein contains the grouping [NR₇-(A′)]but not the grouping —[NR₈-(A′″)]. If the radical R7 now stands for a grouping of the formula (III), the pretreatment agent (a) contains an organic silicon compound with 3 reactive silane groups.

In a further preferred embodiment, a process as contemplated herein is exemplified in that in step (1) one or more organic C₁-C₆ alkoxy silanes of the formula (II) are reacted with water

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
e and f both stand for the number 1,
g and h both stand for the number 0,
A and A′ independently represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

In a further preferred embodiment, a process as contemplated herein is exemplified in that in step (1) one or more organic C₁-C₆ alkoxy silanes of formula (II) are mixed or reacted with water, where
e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂),
   and
R7 represents a hydrogen atom, a methyl group, a 2-hydroxyethyl group, a 2-alkenyl group, a 2-aminoethyl group or a group of formula (III).

Organic silicon compounds of the formula (II) which are well suited for solving the problem as contemplated herein are

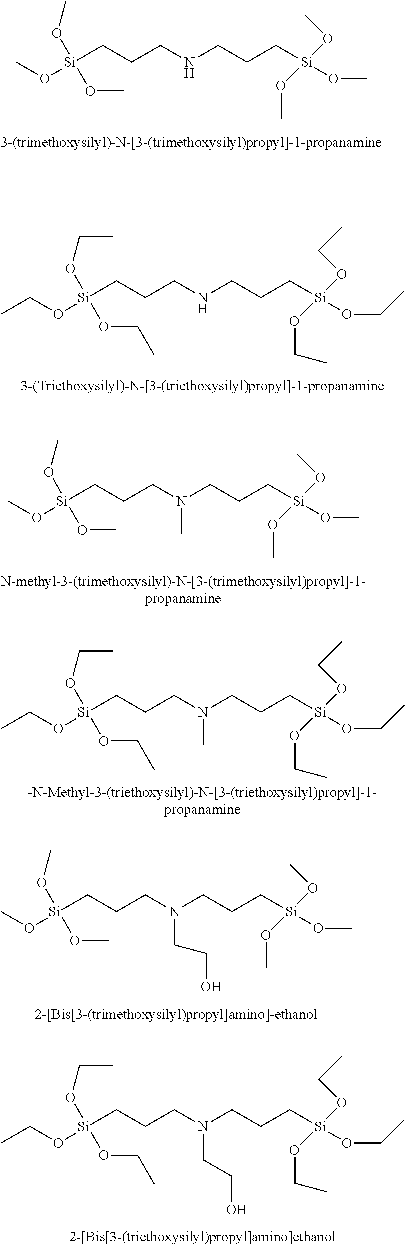

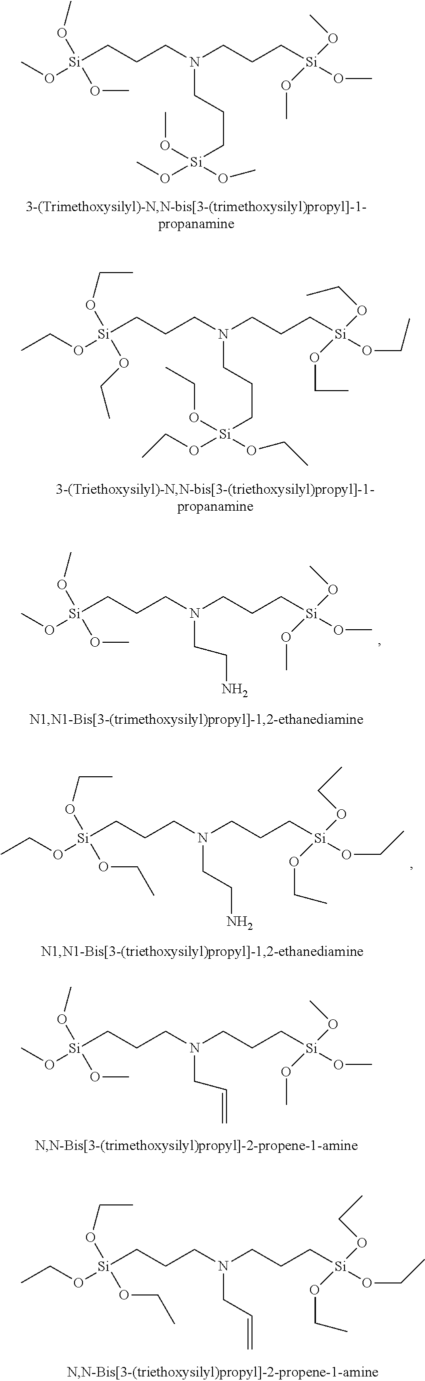

The organic silicon compounds of formula (II) are commercially available. Bis(trimethoxysilylpropyl)amines with the CAS number 82985-35-1 can be purchased from Sigma-Aldrich®. Bis[3-(triethoxysilyl)propyl]amines with the CAS number 13497-18-2 can be purchased from Sigma-Aldrich®, for example. N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich®or Fluorochem®. 3-(triethoxysilyl)-N,N-bis[3-(triethoxysilyl)propyl]-1-propanamine with the CAS number 18784-74-2 can be purchased for example from Fluorochem® or Sigma-Aldrich®.

In a further preferred embodiment, a process as contemplated herein is exemplified in that in step (1) one or more organic C₁-C₆ alkoxy silanes of formula (II) selected from the group of
3-(trimethoxysilyl)-N-[3-(trimethoxysilyl) propyl]-1-propanamine
3-(Triethoxysilyl)-N-[3-(triethoxysilyl) propyl]-1-propanamine
N-methyl-3-(trimethoxysilyl)-N-[3-(trimethoxysilyl) propyl]-1-propanamine
N-Methyl-3-(triethoxysilyl)-N-[3-(triethoxysilyl) propyl]-1-propanamine
2-[Bis[3-(trimethoxysilyl) propyl]amino]-ethanol
2-[bis[3-(triethoxysilyl) propyl]amino]ethanol
3-(Trimethoxysilyl)-N,N-bis[3-(trimethoxysilyl) propyl]-1-propanamine
3-(Triethoxysilyl)-N,N-bis[3-(triethoxysilyl) propyl]-1-propanamine
N1,N1-bis[3-(trimethoxysilyl) propyl]-1,2-ethanediamine,
N1,N1-bis[3-(triethoxysilyl) propyl]-1,2-ethanediamine,
N,N-bis[3-(trimethoxysilyl)propyl]-2-propen-1-amine and/or
N,N-bis[3-(triethoxysilyl)propyl]-2-propene-1-amine,
are reacted with water or mixed with water.

In further dyeing trials, it has also been found to be particularly advantageous if at least one organic C₁-C₆ alkoxy silane of the formula (IV) was used in the process as contemplated herein

R₉Si(OR₁₀)ₖ(R₁₁)ₘ (IV).

The compounds of formula (IV) are organic silicon compounds selected from silanes having one, two or three silicon atoms, wherein the organic silicon compound comprises one or more hydrolysable groups per molecule.

The organic silicon compound(s) of formula (IV) may also be referred to as silanes of the alkyl-C₁-C₆-alkoxy-silane type,

R₉Si(OR₁₀)ₖ(R₁₁)ₘ (IV),

where
R₉ represents a C₁-C₁₂ alkyl group,
R₁₀ represents a C₁-C₆ alkyl group,
R₁₁ represents a C₁-C₆ alkyl group
k is an integer from 1 to 3, and
m stands for the integer 3-k.

In a further embodiment, a particularly preferred method as contemplated herein is exemplified by the
(1) Mixing one or more organic C₁-C₆ alkoxy silanes of formula (IV) with water,

R₉Si(OR₁₀)ₖ(R₁₁)ₘ (IV),

where
R₉ represents a C₁-C₁₂ alkyl group,
R₁₀ represents a C₁-C₆ alkyl group,
R₁₁ represents a C₁-C₆ alkyl group
k is an integer from 1 to 3, and
m stands for the integer 3-k.

In the organic C₁-C₆ alkoxy silanes of formula (IV), the R₉ radical represents a C₁-C₁₂ alkyl group. This C₁-C₁₂ alkyl group is saturated and can be linear or branched. Preferably R₉ stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferred, R₉ stands for a methyl group, an ethyl group or an n-octyl group.

In the organic silicon compounds of formula (IV), the radical R₁₀ represents a C₁-C₆ alkyl group. Preferably R10 stands for a methyl group or an ethyl group.

In the organic silicon compounds of formula (IV), the radical Rn represents a C₁-C₆ alkyl group. Preferably R11 stands for a methyl group or an ethyl group.

Furthermore, k stands for a whole number from 1 to 3, and m stands for the whole number 3-k. If k stands for the number 3, then m is equal to 0. If k stands for the number 2, then m is equal to 1. If k stands for the number 1, then m is equal to 2.

Dyeing's with the best wash fastnesses could be obtained if at least one organic silicon compound of formula (IV), in which the radical k represents the number 3, was used in the preparation of the preparation as contemplated herein. In this case the remainder m stands for the number 0.

Organic silicon compounds of the formula (IV) which are particularly suitable for solving the problem as contemplated herein are

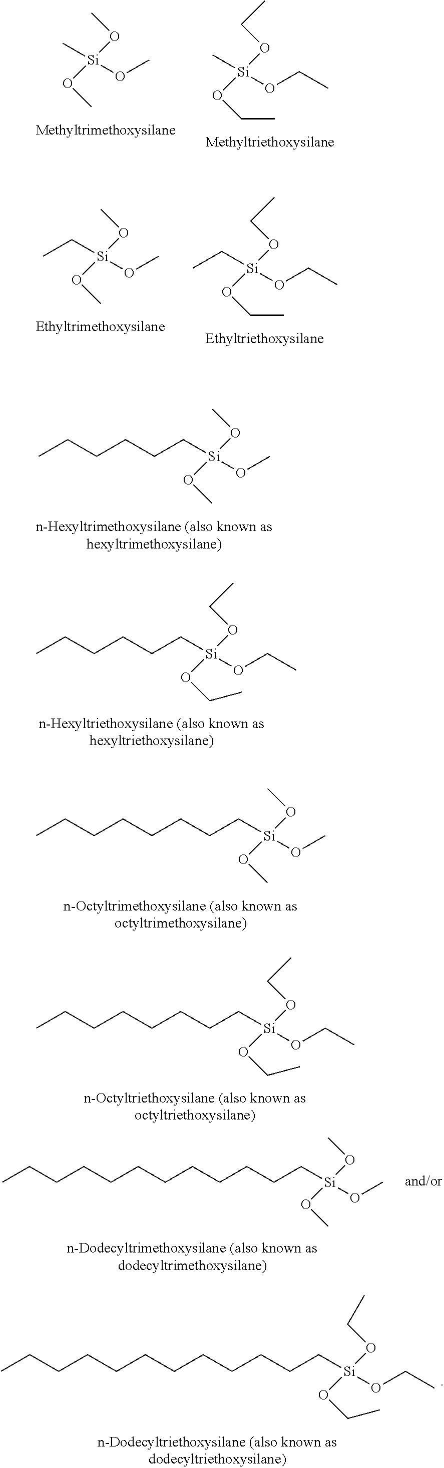

In a further preferred embodiment, a process as contemplated herein is exemplified in that in step (1) one or more organic C₁-C₆ alkoxy silanes of formula (IV) selected from the group of

Methyltrimethoxysilane

Methyltriethoxysilane

Ethyltrimethoxysilane

Ethyltriethoxysilane

Hexyltrimethoxysilane

Hexyltriethoxysilane

Octyltrimethoxysilane

Octyltriethoxysilane

Dodecyltrimethoxysilane and/or

Dodecyltriethoxysilane,

and are mixed with water or reacted with water.

Furthermore, it has been found to be particularly preferred to mix at least one organic C₁-C₆ alkoxy silane of the formula (I) and at least one organic C₁-C₆ alkoxy silane of the formula (IV) with water in step (1) of the process as contemplated herein.

The process as contemplated herein can be carried out in a reaction vessel or reactor suitable for this purpose. Depending on the desired approach size, various prior art models are known and commercially available for this purpose.

For example, the reaction of the organic C₁-C₆ alkoxy silanes with water can be carried out in a reaction vessel or a reactor, preferably a double-walled reactor, a reactor with an external heat exchanger, a tubular reactor, a reactor with a thin-film evaporator, a reactor with a falling-film evaporator, and/or a reactor with an attached condenser.

In another particularly preferred embodiment, a process as contemplated herein is exemplified by:

(1) Mixing one or more organic C₁-C₆ alkoxy silanes with water in a reaction vessel or reactor, preferably in a double-wall reactor, a reactor with an external heat exchanger, a tubular reactor, a reactor with a thin-film evaporator, a reactor with a falling-film evaporator and/or a reactor with an attached condenser.

A reaction vessel that is very suitable for smaller preparations is, for example, a glass flask commonly used for chemical reactions with a capacity of 1 liter, 3 liters or 5 liters, such as a 3-liter single-neck or multi-neck flask with ground joints.

A reactor is a confined space (container, vessel) that has been specially designed and manufactured to allow certain reactions to take place and be controlled under defined conditions.

For larger approaches, it has proven advantageous to carry out the reaction in reactors made of metal. Typical reactors may include, for example, a 10-liter, 20-liter, or 50-liter capacity. Larger reactors for the production area can also include fill volumes of 100-liters, 500-liters, or 1000-liters.

Double-wall reactors have two reactor shells or reactor walls, with a tempering fluid circulating in the area between the two walls. This enables particularly good adjustment of the temperature to the required values.

The use of reactors, in particular double-walled reactors with an enlarged heat exchange surface, has also proven to be particularly suitable, whereby the heat exchange can take place either through internal installations or using an external heat exchanger.

Corresponding reactors are, for example, laboratory reactors from the company IKA®. In this context, the models “LR-2.ST” or the model “magic plant” can be mentioned.

Other reactors that can be used are reactors with thin-film evaporators, since this allows particularly good heat dissipation and thus particularly precise temperature control. Thin film evaporators are alternatively referred to as thin film evaporators. Thin film evaporators can be purchased commercially from Asahi Glassplant® Inc. for example.

In reactors with falling film evaporators, evaporation generally takes place in a tube, i.e., the liquid to be evaporated (i.e., in this case, the C₁-C₆ alcohols to be removed in step (2)) flow as a continuous liquid film. Reactors with falling film evaporators are also commercially available from various suppliers.

The reaction of the organic C₁-C₆ alkoxy silanes with water, which takes place in step (1), can occur in different ways. The reaction starts as soon as the C₁-C₆ alkoxy silanes meet water by mixing. One possibility is to place the desired amount of water in the reaction vessel or reactor and then add that or the C₁-C₆ alkoxy silanes.

In a further embodiment, it is also possible to first introduce the organic C₁-C₆ alkoxy silane(s) into the reaction vessel or reactor and then add the desired amount of water.

As soon as C₁-C₆ alkoxy silanes and water come into contact, an exothermic hydrolysis reaction takes place according to the following scheme (reaction scheme using the example of 3-aminopropyltriethoxysilane):

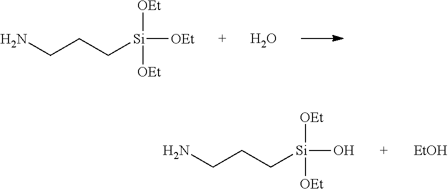 [SMILES: "EtOH" ; CCO[Si](CCCN)(OCC)OCC ; O ; CCO[Si](O)(CCCN)OCC]

Depending on the number of hydrolysable C₁-C₆ alkoxy groups per silane molecule, the hydrolysis reaction can also occur several times per C₁-C₆ alkoxy silane used:

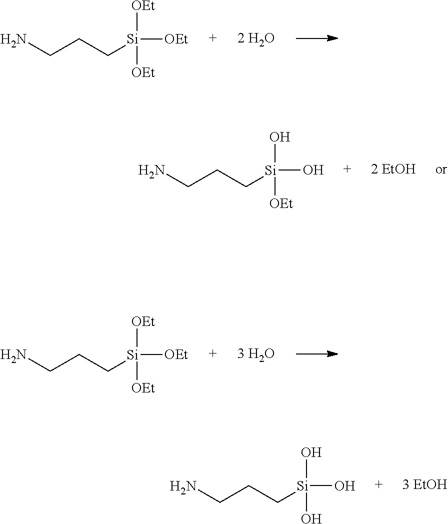 [SMILES: CCO[Si](CCCN)(OCC)OCC ; CCO[Si](O)(O)CCCN ; CCO[Si](CCCN)(OCC)OCC ; NCCC[Si](O)(O)O]

Since the hydrolysis reaction is exothermic, it has been found to be particularly advantageous to stir or mix the reaction mixture of water and organic C₁-C₆ alkoxy silanes for improved heat dissipation.

The water can be added continuously, in partial quantities or directly as a total quantity. To ensure adequate temperature control, the reaction mixture is preferably cooled and/or the amount and rate of water added is adjusted. Depending on the amount of silanes used, the addition and reaction can take place over a period of about 2 minutes to about 72 hours.

For the preparation of agents that produce a particularly good coating on the keratin material, it has been found to be explicitly quite preferred to use water in a sub-stoichiometric amount in step (1). In this case, the amount of water used is below the amount that would theoretically be required to hydrolyze all the hydrolysable C₁-C₆ alkoxy groups present on the Si atoms, i.e., the alkoxysilane groups. Partial hydrolysis of the organic C₁-C₆ alkoxy silanes is therefore particularly preferred.

The stoichiometric ratio of water to the organic C₁-C₆ alkoxy silanes can be defined by the amount of substance equivalent water (S-W), these are calculated according to the following formula:

S−W=mol(water)/(mol(silanes)×n(alkoxy))
with
S-W=molar equivalent water
mol(water)=molar quantity of water used
mol(silanes)=total molar amount of C₁-C₆ alkoxy silanes used in the reaction
n(alkoxy)=number of C₁-C₆ alkoxy groups per C₁-C₆ alkoxy silane

In other words, the molar equivalent of water is the molar ratio of the molar amount of water used to the total molar number of hydrolysable C₁-C₆ alkoxy groups present on the C₁-C₆ alkoxysilanes used.

In another very particularly preferred embodiment, a method as contemplated herein is exemplified by the
(1) Mixing the organic C₁-C₆ alkoxy silanes with from about 0.10 to about 0.80 molar equivalents of water (S-W), preferably from about 0.15 to about 0.70, more preferably from about 0.20 to about 0.60, and most preferably from about 0.25 to about 0.50 molar equivalents of water,
where the molar equivalents of water are calculated according to the formula

S−W=mol(water)/(mol(silane)×n(alkoxy))
with
S-W=molar equivalent water
mol(water)=molar quantity of water used
mol(silanes)=total molar amount of C₁-C₆ alkoxy silanes used in the reaction
n(alkoxy)=number of C₁-C₆ alkoxy groups per C₁-C₆ alkoxy silane

### Example

In a reaction vessel, 20.0 g of 3-aminopropyltriethoxsilane (C₉H₂₃NO₃Si=221.37 g/mol) and 50.0 g of methyltrimethoxysilane (C₄H₁₂O₃Si=136.22 g/mol) were mixed.

20.0 g 3-aminopropyltriethoxsilane=0.0903 mol (3 hydrolysable alkoxy groups per molecule)

50.0 g methyltrimethoxysilane=0.367 mol (3 hydrolysable alkoxy groups per molecule)

Then, 10.0 g of water (18.015 g/mol) was added with stirring. 10.0 g water=0.555 mol

molar equivalent water=0.555 mol/[1(3×0.090 mol)+(3×0.367 mol)]=0.40

In this reaction, the C₁-C₆ alkoxysilanes used were reacted with 0.40 molar equivalents of water.

To produce particularly high-performance keratin treatment agents, maintaining specific temperature ranges has proven to be quite advantageous in step (1).

In this context, it was found that a minimum temperature of 20° C. in step (1) is particularly well suited to allow the hydrolysis to proceed at a sufficiently high rate and to ensure efficient reaction control.

On the other hand, however, heating of the reaction mixture to temperatures above about 70° C. should be avoided. If the production is carried out at too high temperatures, an undesirable or excessive polymerization or condensation reaction will probably take place at this point, resulting in the inability to form a film adhering to the keratin material during subsequent application of the agent. When using an agent produced at too high temperatures in a dyeing process, it was therefore no longer possible to achieve sufficiently high color intensities.

For these reasons, the reaction of the C₁-C₆ organic alkoxy silane(s) with water in step (1) of the process should be carried out at a temperature of about 20 to about 70° C.

The temperature range given here refers to the temperature to which the mixture of C₁-C₆ alkoxy silanes and water should be adjusted. This temperature can be measured, for example, by a calibrated thermometer protruding into this mixture. Preferably, the reaction of one or more organic C₁-C₆ alkoxy silanes with water occurs at a temperature of from about 20° C. to about 70° C., preferably from about 20 to about 65° C., more preferably from about 20 to about 60° C., still more preferably from about 20 to about 55° C., still more preferably from about 20 to about 50° C., and most preferably from about 20 to about 45° C.

In another very particularly preferred embodiment, a method as contemplated herein is exemplified by the

(1) Mixing one or more organic C₁-C₆ alkoxy silanes with water at a temperature of from about 20° C. to about 70° C., preferably from about 20 to about 65° C., more preferably from about 20 to about 60° C., still more preferably from about 20 to about 55° C., still more preferably from about 20 to about 50° C., and most preferably from about 20 to about 45° C.

Adjustment of the preferred and particularly preferred temperature ranges can be accomplished by tempering the reaction vessel or reactor. For example, the reaction vessel or reactor may be surrounded from the outside by a temperature control bath, which may be a water bath or silicone oil bath, for example.

If the reaction is carried out in a double-walled reactor, a temperature-controlled liquid can also be passed through the space formed by the two walls surrounding the reaction chamber.

It may be further preferred that there is no active heating of the reaction mixture and that any increase in temperature above ambient is caused only by the exotherm of the hydrolysis in step (1). If the exothermic reaction process heats the reaction mixture in step (1) too much, it must be cooled again.

The reaction of the organic C₁-C₆ alkoxy silanes with water preferably takes place at normal pressure, i.e., at a pressure of about 1013 mbar (1013 hPa).

### Removal of the C₁-C₆ Alcohols Liberated in Step (1) from the Reaction Mixture

Step (2) of the process as contemplated herein comprises the partial or complete removal from the reaction mixture of the C₁-C₆ alcohols released by the reaction in step (1).

Here it has been found essential to remove the C₁-C₆ alcohols as completely as possible from the reaction mixture so that the total content of C₁-C₆ alcohols in the final preparation obtained after step (4) is below a value of about 8.0% by weight.

In comparative dyeing trials, it was found that the presence of excessive residual amounts of C₁-C₆ alcohols had a negative effect on dyeing performance and color intensity when the preparation was subsequently applied to the keratin material.

As previously described, the hydrolysis of the C₁-C₆ alkoxysilanes releases the corresponding C₁-C₆ alcohols, which can now be removed from the reaction mixture in step (2) and thus removed from the reaction equilibrium.

Since the C₁-C₆ alcohols can be removed from the reaction mixture only after their release occurring in step (1), step (2) of the process preferably occurs after step (1). Here, the removal of the C₁-C₆ alcohols can be done directly after the hydrolysis in step (1). Alternatively, however, a cosmetic ingredient can be added first (corresponding to step (3) of the process as contemplated herein) and the removal of the C₁-C₆ alcohols (step (2)) can be carried out subsequently.

Alternatively, in various embodiments, the performance of step (2) may be performed simultaneously with the hydrolysis in step (1). In such embodiments, the removal of the C₁-C₆ alcohols is already started before the water is added, at the start of the addition or after about 5-20 wt. % of the planned total amount of water has been added, i.e., the distillation is started—optionally under pressure reduction.

Due to the removal of the C₁-C₆ alcohols, the reaction equilibrium is shifted in favor of a condensation reaction in which the Si—OH groups present on the (partially) hydrolyzed C₁-C₆ alkoxysilanes can react with further Si—OH groups or with further C₁-C₆ alkoxy-silane groups with elimination of water.

Such a reaction may proceed, for example, according to the following scheme:

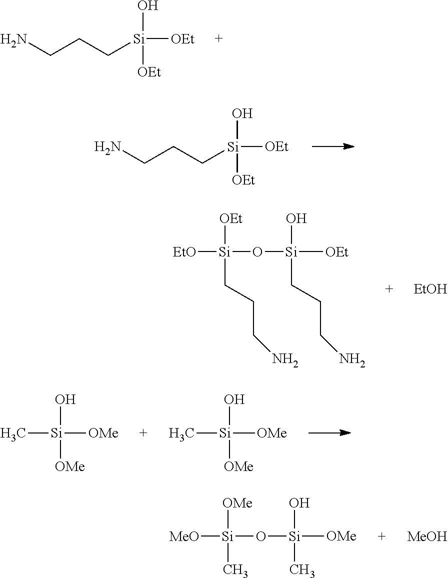

Both partially hydrolyzed and fully hydrolyzed C₁-C₆ alkoxysilanes can participate in the condensation reaction, undergoing condensation with not yet reacted, partially or also fully hydrolyzed C₁-C₆ alkoxysilanes.

In the exemplary reaction scheme above, condensation to a dimer is shown, but condensation to oligomers with multiple silane atoms is also possible and preferred.

In addition, condensation of C₁-C₆ alkoxysilanes of different structures is also possible; for example, the C₁-C₆ alkoxysilanes of formula (I) can condense with the C₁-C₆ alkoxysilanes of formula (IV).

If the released C₁-C₆ alcohols are not removed from the reaction mixture to a sufficient extent in step (2) of the process, the reaction equilibrium shown above can presumably shift back to the side of the monomeric compounds. This back reaction prevents the formation of oligomeric silane condensates with sufficiently high molecular weight, which results in too low color intensities and poorer durability of the formed film or coating when the formulations are later applied to the keratin material.

In the process as contemplated herein, the C₁-C₆ alcohols released are removed as completely as possible. The complete removal of all C₁-C₆ alcohols is difficult to realize, since small residues of C₁-C₆ alcohols will always remain in the reaction mixture, especially if the reaction mixture is not to be heated too much. However, it is essential to the present disclosure to remove all C₁-C₆ alcohols as completely as possible in step (2) of the process, in that the total content of C₁-C₆ alcohols in the final preparation obtained in step (4)—based on the total weight of the preparation—can be kept below about 10.0% by weight.

The extent of the condensation reaction is partly determined by the amount of water added in step (1). Preferably, the amount of water is such that the condensation is a partial condensation, where “partial condensation” or “partial condensation” in this context means that not all the condensable groups of the silanes presented react with each other, so that the resulting organic silicon compound still has on average at least one hydrolysable/condensable group per molecule.

Furthermore, it has been found that the temperature at which the C₁-C₆ alcohols are removed from the reaction mixture in step (2) can also be a significant influencing factor regarding the performance of the subsequent hair treatment product.

In this context, it is suspected that excessively hot temperatures above about 70° C. shift condensation towards high molecular weight products that are too large to be deposited as a closed and resistant film on the keratin material during subsequent keratin treatment. For this reason, it is particularly preferred to maintain a temperature range of about 20 to about 70° C. when removing the C₁-C₆ alcohols from the reaction mixture.

It is particularly preferred to maintain a temperature range of from about 20° C. to about 70° C., preferably from about 20 to about 65° C., more preferably from about 20 to about 60° C., still more preferably from about 20 to about 55° C., still more preferably from about 20 to about 50° C., and most preferably from about 20 to about 45° C. when removing the C₁-C₆ alcohols released by the reaction in step (1).

In step (2), the specified temperature range again refers to the temperature to which the reaction mixture must be adjusted while the C₁-C₆ alcohols are removed from the reaction mixture. This temperature can also be measured, for example, by a calibrated thermometer protruding into this mixture.

In another very particularly preferred embodiment, a method as contemplated herein is exemplified by the
(2) partial or complete removal of the C₁-C₆ alcohols released by the reaction in step (1) from the reaction mixture at a temperature of from about 20° C. to about 70° C., preferably from about 20 to about 65° C., more preferably from about 20 to about 60° C., still more preferably from about 20 to about 55° C., still more preferably from about 20 to about 50° C., and most preferably from about 20 to about 45° C.

In step (2) of the process, the setting of the preferred temperature ranges as contemplated herein can be carried out, for example, by heating or cooling the reaction vessel or reactor, for example by placing the reaction vessel in a heating mantle, or by surrounding the reaction vessel from the outside with a temperature-controlled bath. The tempering bath can be, for example, a water bath or silicone oil bath.

If the reaction is carried out in a double-walled reactor, a temperature-controlled liquid can also be passed through the space formed by the two walls surrounding the reaction chamber.

In step (2) of the process, to ensure the most complete removal of the released C₁-C₆ alcohols without exceeding the preferred temperature range, the C₁-C₆ alcohols are preferably removed under reduced pressure (compared to normal pressure). In this context, it has proved particularly advantageous to distill the C₁-C₆ alcohols from the reaction mixture using a distillation unit. During this distillation, a pressure of about 10 to about 900 mbar is preferably set, more preferably of about 10 to about 800 mbar, still more preferably of about 10 to about 600 mbar and most preferably of about 10 to about 300 mbar.

Vacuum distillation is a common chemical process for which standard commercially available vacuum pumps and distillation apparatus can be used. The distillation apparatus can be in the form of an attachment on the reaction vessel or reactor.

In another very particularly preferred embodiment, a method as contemplated herein is exemplified by the

(2) partial or complete removal of the C₁-C₆ alcohols released by the reaction in step (1) from the reaction mixture by distillation at a pressure of from about 10 to about 900 mbar, more preferably from about 10 to about 800 mbar, still more preferably from about 10 to about 600 mbar and most preferably from about 10 to about 300 mbar.

Another way to ensure the most complete removal of the C₁-C₆ alcohols released by the reaction in step (1) is to carry out the distillation over certain minimum periods of time. The duration of the distillation is partly determined by the preparation size selected in the process as contemplated herein. However, with a usual batch size of up to about 50 kg, preferably of up to about 20 kg, it may be of advantage to carry out the distillation, with adjustment of the above-mentioned temperature and pressure conditions, over a period of at least about 90 minutes, preferably of at least about 120 minutes, further preferably of at least about 150 minutes and very particularly preferably at least about 180 minutes. After, for example, about 300 minutes have elapsed, distillation is then complete.

In another very particularly preferred embodiment, a method as contemplated herein is exemplified by the
(2) partial or complete removal of the C₁-C₆ alcohols released by the reaction in step (1) from the reaction mixture by distillation over a period of about 90 to about 300 minutes, preferably about 120 to about ‘300 minutes, more preferably about 150 to about 300 minutes and most preferably about 180 to about 300 minutes.

Following vacuum distillation, the volatile alcohols and, if necessary, distilled water can be condensed and collected as liquid distillate in a receiver. Distillation can optionally be carried out with cooling of the evaporated alcohols/water by employing a cooler. The reduced pressure can be generated by employing common processes known in the prior art, typically with a vacuum pump.

As already described, C₁-C₆-alkoxysilanes carrying methoxy silane or ethoxy silane groups, di- and trimethoxy- and -ethoxy silanes, especially preferably trimethoxy- or triethoxysilane, are very preferably used in the process as contemplated herein. These have the advantage that methanol and ethanol are released during hydrolysis and condensation, respectively, which can be easily removed from the reaction mixture by vacuum distillation due to their boiling points.

### Addition of One or More Cosmetic Ingredients in Step (3)

As an optional step (3), the process as contemplated herein comprises the addition of one or more cosmetic ingredients.

In accordance with the proviso that the total content of C₁-C₆ alcohols in the preparation obtained in step (4)—based on the total weight of the preparation—must be below about 10.0% by weight, preferably no C₁-C₆ alcohols are added in step (3).

In step (3) of the method, no C₁-C₆ alcohol selected from the group of methanol, ethanol, n-propanol, isopropanol, 1-butanol, 2-butanol, 1-pentanol, 2-pentanol, 3-pentanol, 1-hexanol, 2-hexanol, 3-hexanol, ethylene glycol (1,2-ethanediol), 1,2-propanediol, 1,3-propanediol and glycerol is added to the preparation.

The cosmetic ingredients that may optionally be used in step (3) may be any suitable ingredients to impart further beneficial properties to the product. For example, in step (3) of the process, cosmetic ingredients from the group of solvents other than C₁-C₆ alcohols, thickening or film-forming polymers, surface-active compounds from the group of nonionic, cationic, anionic, or zwitterionic/amphoteric surfactants, coloring compounds from the group of pigments, direct dyes, oxidation dye precursors, fatty components from the group of C₅-C₃₀ fatty alcohols, hydrocarbon compounds, fatty acid esters, acids and bases belonging to the group of pH regulators, perfumes, preservatives, plant extracts and protein hydrolysates may be added.

In a further preferred embodiment, a method as contemplated herein is exemplified by the
(3) Addition of one or more cosmetic ingredients selected from the group of solvents other than C₁-C₆ alcohols, polymers, surface-active compounds, coloring compounds, fatty components, pH regulators, perfumes, preservatives, plant extracts and protein hydrolysates.

In another very particularly preferred embodiment, a method as contemplated herein is exemplified by the
(3) Addition of one or more cosmetic ingredients selected from the group of polymers, surface-active compounds, coloring compounds, lipid components, pH regulators, perfumes, preservatives, plant extracts and protein hydrolysates.

The selection of these other substances will be made by the specialist according to the desired properties of the agents. Regarding other optional components and the quantities of these components used, explicit reference is made to the relevant manuals known to the specialist.

In this context, it has proven to be particularly preferred to use a cosmetic ingredient in step (3) which further improves the stability, in particular the storage stability, of the keratin treatment agent. In this context, the addition (3) of one or more cosmetic ingredients selected from the group of hexamethyldisiloxane, octamethyltrisiloxane, decamethyltetrasiloxane, hexamethylcyclotrisiloxane, octamethylcyclotetrasiloxane and/or decamethylcyclopentasiloxane has been shown to be particularly beneficial in terms of increasing the stability of the composition.

In another very particularly preferred embodiment, a method as contemplated herein is exemplified by the
(3) Addition of one or more cosmetic ingredients selected from the group of hexamethyldisiloxane, octamethyltrisiloxane, decamethyltetrasiloxane, hexamethylcyclotrisiloxane, octamethylcyclotetrasiloxane and/or decamethylcyclopentasiloxane.

Hexamethyldisiloxane has the CAS number 107-46-0 and can be purchased commercially from Sigma-Aldrich®, for example.

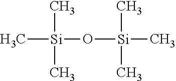

Octamethyltrisiloxane has the CAS number 107-51-7 and is also commercially available from Sigma-Aldrich®.

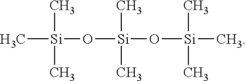

Decamethyltetrasiloxane carries the CAS number 141-62-8 and is also commercially available from Sigma-Aldrich®.

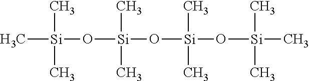

### Hexamethylcyclotrisiloxane has the CAS No. 541-05-9.

### Octamethylcyclotetrasiloxane has the CAS No. 556-67-2.

### Decamethylcyclopentasiloxane has the CAS No. 541-02-6.

### Filling the Preparation into a Packaging Unit (4)

In step (4) of the process as contemplated herein, the preparation obtained after steps (1) and (2)—and optionally after the optional step (3)—is filled into a packaging unit.

The packaging unit can be a final packaging from which the user takes the agent for treatment of the keratin materials. Suitable end-packages include a bottle, a tube, a jar, a can, a sachet, an aerosol pressure container, and/or a non-aerosol pressure container. In this regard, these final packages may contain the keratin treatment agents in quantities sufficient for one, or if necessary, several applications. Preference is given to filling in a quantity sufficient for a single application.

Further, however, the preparation in step (4) may also be filled into an intermediate package, which may be, for example, a canister or a hobbock. Filling into an intermediate package is particularly suitable if the reaction vessel or reactor in which the process as contemplated herein was carried out and the filling plant in which filling into the final package takes place are physically separated.

In another very particularly preferred embodiment, a method as contemplated herein is exemplified by the
(4) Filling the preparation into a bottle, tube, jar, can, sachet, aerosol pressure container, non-aerosol pressure container, canister, or hobbock.

The packaging units may be common, standard, commercially available containers used in cosmetics.

### Total Content of C₁-C₆ Alcohols in the Preparation Obtained in Step (4)

When applying the preparation produced by the process as contemplated herein on the keratin material, the production of a stable, coherent, and uniform coating is the basic requirement for achieving the desired application properties. Intense and long-lasting colorations can be obtained especially if the colorant compounds can be integrated into an appropriately resistant coating. As previously written, it is essential for this purpose to keep the content of C₁-C₆ alcohols in the final preparations obtained after step (4) as low as possible. For this reason, there is a requirement that the total content of C₁-C₆ alcohols in the preparation obtained in step (4)—based on the total weight of the preparation—is below about 10.0% by weight.

The preparation obtained after step (4), which is determined by its maximum content of C₁-C₆ alcohols, is the preparation that was filled into a packaging unit after its preparation. Here, the determination and/or measurement of the content of C₁-C₆ alcohols as contemplated herein is made within about 24 hours after bottling. The indication of the maximum amount of C₁-C₆ alcohols contained in the preparation in percent by weight refers to the total weight of the preparation as it is after filling in the packaging unit according to step (4).

For the purposes of the present disclosure, C₁-C₆ alcohols are alcohols having one or more hydroxy groups comprising from 1 to 6 carbon atoms. These alcohols can be linear or branched, saturated or mono- or polyunsaturated. By C₁-C₆ mono-alcohols are meant the alcohols from the group of methanol, ethanol, n-propanol, isopropanol, 1-butanol, 2-butanol, 1-pentanol, 2-pentanol, 3-pentanol, 1-hexanol, 2-hexanol and 3-hexanol. C₁-C₆ alcohols with two hydroxyl groups include ethylene glycol, 1,2-propanediol and 1,3-propanediol. For example, a C₁-C₆ alcohol with three hydroxyl groups is glycerol.

To comply with these weight specifications, on the one hand the C₁-C₆ alcohols must be removed as completely as possible from the reaction mixture in step (2), and on the other hand no corresponding C₁-C₆ alcohols may be added to the preparation in step (3).

With preparations whose total content of C₁-C₆ alcohols was about 10.0% by weight, dyeing's with sufficiently high color intensity could be obtained when applied to the keratin material.

However, even better results were obtained if the total content of C₁-C₆ alcohols in the preparation obtained after step (4)—based on the total weight of the preparation—could be reduced to a value below about 9.0% by weight, preferably below about 8.0% by weight, more preferably below about 7.0% by weight, even more preferably below about 6.0% by weight and most preferably below about 5.0% by weight.

In another very particularly preferred embodiment, a process as contemplated herein is exemplified in that the total content of C₁-C₆ alcohols in the preparation obtained after step (4)—based on the total weight of the preparation—is below about 9.0% by weight, preferably below about 8.0% by weight, more preferably below about 7.0% by weight, still more preferably below about 6.0% by weight and very particularly preferably below about 5.0% by weight.

In another very particularly preferred embodiment, a process as contemplated herein is exemplified in that the total content of C₁-C₆ alcohols in the preparation obtained after step (4)—based on the total weight of the preparation—is below about 10.0 wt. %, preferably below about 9.0 wt. % by weight, further preferably below about 8.0% by weight, still further preferably below about 7.0% by weight, still further preferably below about 6.0% by weight, and very particularly preferably below about 5.0% by weight, wherein the determination of the content of C₁-C₆ alcohols is carried out within about 24 hours after the filling of the preparation into a packaging unit (4).

In other words, a preferred process as contemplated herein is exemplified in that the total content of C₁-C₆ alcohols in the preparation within about 24 hours after filling into a packaging unit (4)—based on the total weight of the preparation—is below about 10.0% by weight, preferably below about 9.0% by weight, more preferably below about 8.0% by weight, even more preferably below about 7.0% by weight, even more preferably below about 6.0% by weight, and very particularly preferably below about 5.0% by weight.

The determination of the content of C₁-C₆ alcohols in the preparation obtained according to step (4) can be carried out by various analytical methods. One possibility is measurement by GC-MS. Gas chromatography with mass spectrometry coupling is the coupling of a gas chromatograph (GC) with a mass spectrometer (MS). The overall procedure or instrument coupling is also referred to as GC-MS, GC/MS or GCMS for short

To determine the content of C₁-C₆ alcohols, a sample of the preparation can be analyzed by gas chromatography in a double determination on a non-polar column, for example. Identification of the assigned components can be performed by mass spectrometry using library comparison spectra (e.g., NIST or Wiley). The mean value is formed from each of the double determinations. Quantification can be performed, for example, by employing internal standard calibration (e.g., with methyl isobutyl ketone).

### Water Content of the Preparation Obtained after Step (4)

To be able to provide a formulation that is as stable as possible during storage, the preparation obtained in step (4) preferably has as low a water content as possible. The water content of the product after vacuum distillation is therefore preferably below about 5.0 wt. %, preferably below about 4.0 wt. %, more preferably below about 3.0 wt. %, still more preferably below about 2.0 wt. % and most preferably below about 1.0 wt. %. Here, the water content of the preparation, given in % by weight, is related to the total weight of the preparation obtained after step (4).

In another very particularly preferred embodiment, a process as contemplated herein is exemplified in that the water content in the preparation obtained after step (4)—based on the total weight of the preparation—is below about 5.0% by weight, preferably below about 4.0% by weight, further preferably below about 3.0% by weight, still further preferably below about 2.0% by weight and very particularly preferably below about 1.0% by weight.

In other words, a preferred method as contemplated herein is exemplified in that the water content in the preparation obtained after step (4)—based on the total weight of the preparation—is below about 5.0% by weight, preferably below about 4.0% by weight, further preferably below about 3.0% by weight, still further preferably below about 2.0% by weight and very particularly preferably below about 1.0% by weight, the determination of the water content being carried out within about 24 hours after the filling of the preparation into a packaging unit (4).

Preferably, a process for the preparation of an agent for the treatment of keratinous material, in particular human hair, comprising the following steps:
(1) Mixing one or more organic C₁-C₆ alkoxy silanes with water,
(2) partial or complete removal from the reaction mixture of the C₁-C₆ alcohols released by the reaction in step (1),
(3) optionally addition of one or more cosmetic ingredients, and
(4) Filling of the preparation into a packaging unit,
   exemplified in that
the total content of C₁-C₆ alcohols in the preparation obtained in step (4)—based on the total weight of the preparation—is below about 10.0% by weight, and
the water content in the preparation obtained after step (4)—based on the total weight of the preparation—is below about 5.0% by weight.

Preferably, a process for the preparation of an agent for the treatment of keratinous material, in particular human hair, comprising the following steps:
(1) Mixing one or more organic C₁-C₆ alkoxy silanes with water,
(2) partial or complete removal from the reaction mixture of the C₁-C₆ alcohols released by the reaction in step (1),
(3) optionally addition of one or more cosmetic ingredients, and
(4) Filling of the preparation into a packaging unit,
   exemplified in that
the total content of C₁-C₆ alcohols in the preparation obtained in step (4)—based on the total weight of the preparation—is below about 9.0% by weight, and
the water content in the preparation obtained after step (4)—based on the total weight of the preparation—is below about 4.0% by weight.

Preferably, a process for the preparation of an agent for the treatment of keratinous material, in particular human hair, comprising the following steps:
(1) Mixing one or more organic C₁-C₆ alkoxy silanes with water,
(2) partial or complete removal from the reaction mixture of the C₁-C₆ alcohols released by the reaction in step (1),
(3) optionally addition of one or more cosmetic ingredients, and
(4) Filling of the preparation into a packaging unit,
   exemplified in that
the total content of C₁-C₆ alcohols in the preparation obtained in step (4)—based on the total weight of the preparation—is below about 8.0% by weight, and
the water content in the preparation obtained after step (4)—based on the total weight of the preparation—is below about 3.0% by weight.

Preferably, a process for the preparation of an agent for the treatment of keratinous material, in particular human hair, comprising the following steps:
(1) Mixing one or more organic C₁-C₆ alkoxy silanes with water,
(2) partial or complete removal from the reaction mixture of the C₁-C₆ alcohols released by the reaction in step (1),
(3) optionally addition of one or more cosmetic ingredients, and
(4) Filling of the preparation into a packaging unit,
   exemplified in that
the total content of C₁-C₆ alcohols in the preparation obtained in step (4)—based on the total weight of the preparation—is below about 7.0% by weight, and
the water content in the preparation obtained after step (4)—based on the total weight of the preparation—is below about 2.0% by weight.

Preferably, a process for the preparation of an agent for the treatment of keratinous material, in particular human hair, comprising the following steps:
(1) Mixing one or more organic C₁-C₆ alkoxy silanes with water,
(2) partial or complete removal from the reaction mixture of the C₁-C₆ alcohols released by the reaction in step (1),
(3) optionally addition of one or more cosmetic ingredients, and
(4) Filling of the preparation into a packaging unit, exemplified in that
the total content of C₁-C₆ alcohols in the preparation obtained in step (4)—based on the total weight of the preparation—is below about 6.0% by weight, and
the water content in the preparation obtained after step (4)—based on the total weight of the preparation—is below about 1.0% by weight.

### Sequence of the Process Steps

In one embodiment, the method comprises steps (1), (2), (3) and (4), step (3) being an optional step. Regarding the sequence of the process steps, several embodiments are suitable.

In one embodiment, preferred is a method comprising the steps in the following order:
(1) Reaction of one or more organic C₁-C₆ alkoxy silanes with water,
(2) partial or complete removal from the reaction mixture of the C₁-C₆ alcohols released by the reaction in step (1),
(3) optionally addition of one or more cosmetic ingredients, and
(4) Filling of the preparation into a packaging unit.

This procedure starts with step (1), followed by step (2), followed by step (3), followed by step (4). First, one or more organic C₁-C₆ alkoxy silanes are mixed with water, and the C₁-C₆ alcohols formed in this reaction are removed as completely as possible in step (2). One or more cosmetic ingredients are then added to the reaction mixture, which may be, for example, an aprotic solvent, a pigment, a thickening polymer, or the like (step 3). The preparation is then filled into a packaging unit (step 4).

In a further embodiment, it may be equally preferred to perform the addition of the cosmetic ingredient(s) (3) prior to removal of the C₁-C₆ alcohols in step (2).

In yet another embodiment, preferred is a method comprising the steps in the following order:
(1) Reaction of one or more organic C₁-C₆ alkoxy silanes with water,
(3) if necessary, addition of one or more cosmetic ingredients,
(2) partial or complete removal from the reaction mixture of the C₁-C₆ alcohols released by the reaction in step (1), and
(4) Filling the preparation into a packaging unit.

### pH Values of the Preparations in the Process

In further experiments, it has been found that the pH values possessed by the reaction mixture during steps (1) to (4) of the process as contemplated herein can also have an influence on the condensation reaction. It was found that alkaline pH values in particular stop condensation at the oligomer stage. The more acidic the reaction mixture, the more condensation seems to take place and the higher the molecular weight of the siloxanes formed during condensation. For this reason, it is preferred that the reaction mixture in step (1), (2), (3) and/or (4) has a pH of from about 7.0 to about 12.0, preferably from about 7.5 to about 11.5, more preferably from about 8.5 to about 11.0, and most preferably from about 9.0 to about 11.0.

In another very particularly preferred embodiment, a process as contemplated herein, is exemplified in that the reaction mixture in step (1), (2), (3) and/or (4), after mixing in a weight ratio of about 1:1 with water, has a pH of from about 7.0 to about 12.0, preferably from about 7.5 to about 11.5, more preferably from about 8.5 to about 11.0 and very particularly preferably from about 9.0 to about 11.0.

In another very particularly preferred embodiment, a process as contemplated herein, is exemplified in that the reaction mixture in steps (1) to (6), after mixing in a weight ratio of about 1:1 with water, has a pH of from about 7.0 to about 12.0, preferably from about 7.5 to about 11.5, more preferably from about 8.5 to about 11.0 and very particularly preferably from about 9.0 to about 11.0.

To adjust this alkaline pH, it may be necessary to add an alkalizing agent and/or acidifying agent to the reaction mixture. The pH values for the purposes of the present disclosure are pH values measured at a temperature of about 22° C.

For example, ammonia, alkanolamines and/or basic amino acids can be used as alkalizing agents.

Alkanolamines may be selected from primary amines having a C₂-C₆ alkyl parent bearing at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol.

A particularly preferred embodiment is therefore exemplified in that the agent as contemplated herein contains an alkanolamine selected from 2-aminoethan-1-ol and/or 2-amino-2-methylpropan-1-ol as alkalizing agent. Preferred amino acids are amino carboxylic acids, especially α-(alpha)-amino carboxylic acids and ω-amino carboxylic acids, whereby α-amino carboxylic acids are particularly preferred.

As contemplated herein, basic amino acids are those amino acids which have an isoelectric point pI of greater than about 7.0.

Basic α-amino carboxylic acids contain at least one asymmetric carbon atom. In the context of the present disclosure, both possible enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine, and histidine, especially preferably arginine and lysine. In another particularly preferred embodiment, an agent as contemplated herein is therefore exemplified in that the alkalizing agent is a basic amino acid from the group arginine, lysine, ornithine and/or histidine.

In addition, inorganic alkalizing agents can also be used. Inorganic alkalizing agents usable as contemplated herein are preferably selected from the group formed by sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Particularly preferred alkalizing agents are ammonia, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-Amino-2-methylpropan-2-ol, 3-aminopropan-1,2-diol, 2-amino-2-methylpropan-1,3-diol, arginine, lysine, ornithine, histidine, sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Besides the alkalizing agents described above, experts are familiar with common acidifying agents for fine adjustment of the pH-value. As contemplated herein, preferred acidifiers are pleasure acids, such as citric acid, acetic acid, malic acid, or tartaric acid, as well as diluted mineral acids.

### Agent for the Treatment of Keratinous Material

The process described above allows the preparation of prehydrolyzed or condensed silane blends, which perform exceptionally well when applied to keratinous material.

In principle, the keratin treatment agents produced by this process can be used for various purposes, for example as agents for coloring keratinous material, as agents for caring for keratinous material or as agents for changing the shape of keratinous material.

In another very particularly preferred embodiment, a process as contemplated herein is exemplified in that an agent for coloring keratinous material, for maintaining keratinous material or for changing the shape of keratinous material is prepared, stored, and later applied.

In another very particularly preferred embodiment, a process as contemplated herein is exemplified in that an agent is prepared for coloring keratinous material, for maintaining keratinous material or for changing the shape of keratinous material.

Explicitly, the prepared agents show particularly good suitability when used in a dyeing process.

In another very particularly preferred embodiment, a process as contemplated herein is exemplified in that an agent for coloring keratinous material is prepared.

When used in a dyeing process, at least one colorant compound may be added to the composition, for example in step (3), wherein the colorant compound may be selected from the group of pigments, direct dyes and/or oxidation dye precursors. Here, an agent for coloring keratin material can be obtained which, in addition to the prehydrolyzed/condensed C₁-C₆ alkoxysilanes, also contains the coloring compound(s).

### Agent in Packing Unit

In one embodiment, preparations prepared via the method as contemplated herein can be taken directly from the packaging unit during their application and applied to the keratin material by the user.

A second subject matter of the present disclosure is an agent for treating keratinous material comprising a preparation in a packaging unit prepared according to a process as disclosed in detail in the description of the first subject matter of the present disclosure.

### Multi-Component Packaging Unit (Kit-of-Parts)

In the context of a further embodiment, however, it is also particularly preferred if the preparation prepared according to step (4) of the method is first mixed with a further preparation so that a colorant ready for use is obtained. This ready-to-use colorant is then applied to the keratin materials. This embodiment is particularly preferred when the preparations are used in a dyeing process.

To increase user convenience, all preparations required for the staining process are provided to the user in the form of a multi-component packaging unit (kit-of-parts).

A third object of the present discosure is a multi-component packaging unit (kit-of-parts) for dyeing keratinous material, in particular human hair, which are separately assembled
a first packaging unit containing a cosmetic preparation (A) and
a second packaging unit containing a cosmetic preparation (B),
   where
the cosmetic preparation (A) in the first packaging unit has been produced according to the method as disclosed in detail in the description of the first subject-matter of the present disclosure, and
the cosmetic formulation (B) comprises at least one colorant compound selected from the group of pigments, direct dyes and/or oxidation dye precursors.

Just before application, the two preparations (A) and (B) are then mixed, and this ready-to-use staining agent is then applied to the keratin material.

Furthermore, the multi-component packaging unit as contemplated herein may also comprise a third packaging unit containing a cosmetic preparation (C). Preparation (C) may be, for example, a conditioner, a shampoo, or a pre- or post-treatment agent.

### Coloring Compounds

When the agents prepared via the process as contemplated herein are used in a dyeing process, one or more colorant compounds may be employed. The colorant compound(s) can either be added to the reaction mixture as cosmetic ingredients in step (3) of the process or provided to the user as an ingredient of a separately prepared preparation (B).

The coloring compound or compounds can preferably be selected from pigments, substantive dyes, oxidation dyes, photochromic dyes and thermochromic dyes, particularly preferably from pigments and/or substantive dyes.

Pigments within the meaning of the present disclosure are coloring compounds which have a solubility in water at 25° C. of less than about 0.5 g/L, preferably less than about 0.1 g/L, even more preferably less than about 0.05 g/L. Water solubility can be determined, for example, by the method described below: 0.5 g of the pigment are weighed in a beaker. A stir-fish is added. Then one liter of distilled water is added. This mixture is heated to about 25° C. for about ‘one hour while stirring on a magnetic stirrer. If undissolved components of the pigment are still visible in the mixture after this period, the solubility of the pigment is below about 0.5 g/L. If the pigment-water mixture cannot be assessed visually due to the high intensity of the possibly finely dispersed pigment, the mixture is filtered. If a proportion of undissolved pigments remains on the filter paper, the solubility of the pigment is below about 0.5 g/L.

Suitable color pigments can be of inorganic and/or organic origin.

In a preferred embodiment, a composition as contemplated herein is exemplified in that it comprises at least one colorant compound selected from the group of inorganic and/or organic pigments.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-containing silicates, silicates, metal sulfides, complex metal cyanides, metal sulphates, chromates and/or molybdates. Preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

Colored pearlescent pigments are also particularly preferred colorants from the group of pigments as contemplated herein. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a further preferred embodiment, an agent as contemplated herein is exemplified in that it comprises (b) at least one colorant compound from the group of pigments selected from the group of colored metal oxides, metal hydroxides, metal oxide hydrates, silicates, metal sulfides, complex metal cyanides, metal sulfates, bronze pigments and/or from mica- or mica-based colorant compounds coated with at least one metal oxide and/or a metal oxychloride.

In a further preferred embodiment, a composition as contemplated herein is exemplified in that it comprises (b) at least one colorant compound selected from mica- or mica-based pigments reacted with one or more metal oxides selected from the group of titanium dioxide (CI 77891), black iron oxide (CI 77499), yellow iron oxide (CI 77492), red and/or brown iron oxide (CI 77491, CI 77499), manganese violet (CI 77742), ultramarines (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available under the trade names Rona®, Colorona®, Xirona®, Dichrona® and Timiron® from Merck®, Ariabel® and Unipure® from Sensient®, Prestige® from Eckart® Cosmetic Colors and Sunshine® from Sunstar®.

Particularly preferred color pigments with the trade name Colorona® are, for example:

### Colorona® Copper, Merck®, MICA, CI 77491 (IRON OXIDES)

### Colorona® Passion Orange, Merck®, Mica, CI 77491 (Iron Oxides), Alumina

### Colorona® Patina Silver, Merck®, MICA, CI 77499 (IRON OXIDES), CI 77891 (TITANIUM DIOXIDE)

### Colorona® RY, Merck®, CI 77891 (TITANIUM DIOXIDE), MICA, CI 75470 (CARMINE)

### Colorona® Oriental Beige, Merck®, MICA, CI 77891 (TITANIUM DIOXIDE), CI 77491 (IRON OXIDES)

### Colorona® Dark Blue, Merck®, MICA, TITANIUM DIOXIDE, FERRIC FERROCYANIDE

### Colorona® Chameleon, Merck®, CI 77491 (IRON OXIDES), MICA

### Colorona® Aborigine Amber, Merck®, MICA, CI 77499 (IRON OXIDES), CI 77891 (TITANIUM DIOXIDE)

### Colorona® Blackstar Blue, Merck®, CI 77499 (IRON OXIDES), MICA

### Colorona® Patagonian Purple, Merck®, MICA, CI 77491 (IRON OXIDES), CI 77891 (TITANIUM DIOXIDE), CI 77510 (FERRIC FERROCYANIDE)

### Colorona® Red Brown, Merck®, MICA, CI 77491 (IRON OXIDES), CI 77891 (TITANIUM DIOXIDE)

### Colorona® Russet, Merck®, CI 77491 (TITANIUM DIOXIDE), MICA, CI 77891 (IRON OXIDES)

### Colorona® Imperial Red, Merck®, MICA, TITANIUM DIOXIDE (CI 77891), D&C RED NO. 30 (CI 73360)

### Colorona® Majestic Green, Merck®, CI 77891 (TITANIUM DIOXIDE), MICA, CI 77288 (CHROMIUM OXIDE GREENS)

### Colorona® Light Blue, Merck®, MICA, TITANIUM DIOXIDE (CI 77891), FERRIC FERROCYANIDE (CI 77510)

### Colorona® Red Gold, Merck, MICA®, CI 77891 (TITANIUM DIOXIDE), CI 77491 (IRON OXIDES)

### Colorona® Gold Plus MP 25, Merck®, MICA, TITANIUM DIOXIDE (CI 77891), IRON OXIDES (CI 77491)

### Colorona® Carmine Red, Merck®, MICA, TITANIUM DIOXIDE, CARMINE

### Colorona® Blackstar Green, Merck®, MICA, CI 77499 (IRON OXIDES)

### Colorona® Bordeaux, Merck®, MICA, CI 77491 (IRON OXIDES)

### Colorona® Bronze, Merck®, MICA, CI 77491 (IRON OXIDES)

### Colorona® Bronze Fine, Merck®, MICA, CI 77491 (IRON OXIDES)

### Colorona® Fine Gold MP 20, Merck®, MICA, CI 77891 (TITANIUM DIOXIDE), CI 77491 (IRON OXIDES)

### Colorona® Sienna Fine, Merck®, CI 77491 (IRON OXIDES), MICA

### Colorona® Sienna, Merck®, MICA, CI 77491 (IRON OXIDES)

Colorona® Precious Gold, Merck®, Mica, CI 77891 (Titanium dioxide), Silica, CI 77491(Iron oxides), Tin oxide

### Colorona® Sun Gold Sparkle MP 29, Merck®, MICA, TITANIUM DIOXIDE, IRON OXIDES, MICA, CI 77891, CI 77491 (EU)

Colorona® Mica Black, Merck®, CI 77499 (Iron oxides), Mica, CI 77891 (Titanium dioxide)
Colorona® Bright Gold, Merck®, Mica, CI 77891 (Titanium dioxide), CI 77491(Iron oxides)

### Colorona® Blackstar Gold, Merck®, MICA, CI 77499 (IRON OXIDES)

Other particularly preferred color pigments with the trademark Xirona® are for example:

### Xirona® Golden Sky, Merck®, Silica, CI 77891 (Titanium Dioxide), Tin Oxide

### Xirona® Caribbean Blue, Merck®, Mica, CI 77891 (Titanium Dioxide), Silica, Tin Oxide

### Xirona® Kiwi Rose, Merck®, Silica, CI 77891 (Titanium Dioxide), Tin Oxide

### Xirona® Magic Mauve, Merck®, Silica, CI 77891 (Titanium Dioxide), Tin Oxide.

In addition, particularly preferred color pigments with the trademark Unipure LC® are for example:

### Unipure Red LC® 381 EM, Sensient® CI 77491 (Iron Oxides), Silica

### Unipure Black LC® 989 EM, Sensient®, CI 77499 (Iron Oxides), Silica

### Unipure Yellow LC® 182 EM, Sensient®, CI 77492 (Iron Oxides), Silica

In a further embodiment, the composition or preparation as contemplated herein may also contain one or more colorant compounds selected from the group of organic pigments

The organic pigments as contemplated herein are correspondingly insoluble, organic dyes or color lacquers, which may be selected, for example, from the group of nitroso, nitro-azo, xanthene, anthraquinone, isoindolinone, isoindolinone, quinacridone, perinone, perylene, diketo-pyrrolopyrrole, indigo, thioindigo, dioxazine and/or triarylmethane compounds.

Examples of particularly suitable organic pigments are carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In a further particularly preferred embodiment, a composition as contemplated herein is exemplified in that it comprises at least one colorant compound from the group of organic pigments selected from the group of carmine, quinacridone, phthalocyanine, sorghum, blue pigments having the Color Index numbers Cl 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color paint. In the sense of the present disclosure, the term color lacquer means particles comprising a layer of absorbed dyes, the unit of particle and dye being insoluble under the above-mentioned conditions. The particles can, for example, be inorganic substrates, which can be aluminum, silica, calcium borosilate, calcium aluminum borosilicate or even aluminum.

For example, alizarin color varnish can be used.

Due to their excellent resistance to light and temperature, the use of the pigments as contemplated herein is particularly preferred. It is also preferred if the pigments used have a certain particle size. This particle size leads on the one hand to an even distribution of the pigments in the formed polymer film and on the other hand avoids a rough hair or skin feeling after application of the cosmetic product. As contemplated herein, it is therefore advantageous if the at least one pigment has an average particle size D50 of about 1.0 to about 50 μm, preferably about 5.0 to about 45 μm, preferably about 10 to about 40 μm, about 14 to about 30 μm. The mean particle size D50D₅₀, for example, can be determined using dynamic light scattering (DLS).

The pigment or pigments may be used in an amount of from about 0.001 to about 20% by weight, or from about 0.05 to about 5% by weight, in each case based on the total weight of the composition or preparation as contemplated herein.

As colorant compounds, the compositions as contemplated herein may also contain one or more direct dyes. Direct-acting dyes are dyes that draw directly onto the hair and do not require an oxidative process to form the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at 25° C. of more than about 0.5 g/L and are therefore not to be regarded as pigments. Preferably, the direct dyes within the meaning of the present disclosure have a solubility in water (760 mmHg) at 25° C. of more than about 1.0 g/L. In particular, the direct dyes within the meaning of the present disclosure have a solubility in water (760 mmHg) at 25° C. of more than about 1.5 g/L.

Direct dyes can be divided into anionic, cationic, and nonionic direct dyes.

In a further preferred embodiment, an agent as contemplated herein is exemplified in that it contains at least one anionic, cationic and/or nonionic direct dye as the coloring compound.

In a further preferred embodiment, an agent as contemplated herein is exemplified in that it comprises at least one anionic, cationic and/or nonionic direct dye.

Suitable cationic direct dyes include Basic Blue 7, Basic Blue 26, Basic Violet 2, and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51 Basic Red 76

As non-ionic direct dyes, non-ionic nitro and quinone dyes and neutral azo dyes can be used. Suitable non-ionic direct dyes are those listed under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

Anionic direct dyes are also called acid dyes. Acid dyes are direct dyes that have at least one carboxylic acid group (—COOH) and/or one sulphonic acid group (—SO₃H). Depending on the pH value, the protonated forms (—COOH, —SO₃H) of the carboxylic acid or sulphonic acid groups are in equilibrium with their deprotonated forms (—OO⁻, —SO₃-present). The proportion of protonated forms increases with decreasing pH. If direct dyes are used in the form of their salts, the carboxylic acid groups or sulphonic acid groups are present in deprotonated form and are neutralized with corresponding stoichiometric equivalents of cations to maintain electro neutrality. Acid dyes can also be used in the form of their sodium salts and/or their potassium salts.

The acid dyes within the meaning of the present disclosure have a solubility in water (760 mmHg) at 25° C. of more than about 0.5 g/L and are therefore not to be regarded as pigments. Preferably the acid dyes within the meaning of the present disclosure have a solubility in water (760 mmHg) at 25° C. of more than about 1.0 g/L.

The alkaline earth salts (such as calcium salts and magnesium salts) or aluminum salts of acid dyes often have a lower solubility than the corresponding alkali salts. If the solubility of these salts is below about 0.5 g/L (25° C., 760 mmHg), they do not fall under the definition of a direct dye.

An essential property of acid dyes is their ability to form anionic charges, whereby the carboxylic acid or sulphonic acid groups responsible for this are usually linked to different chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

For example, one or more compounds from the following group can be selected as particularly well suited acid dyes: Acid Yellow 1 (D&C Yellow 7, Citronin A, Ext. D&C Yellow No. 7, Japan Yellow 403, CI 10316, COLIPA n° B001), Acid Yellow 3 (COLIPA n°: C 54, D&C Yellow N° 10, Quinoline Yellow, E104, Food Yellow 13), Acid Yellow 9 (CI 13015), Acid Yellow 17 (CI 18965), Acid Yellow 23 (COLIPA n° C. 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA n° C.015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; nosodiumsalt; Brown No. 201; RESORCIN BROWN; ACID ORANGE 24; Japan Brown 201; D & C Brown No. 1), Acid Red 14 (C.I.14720), Acid Red 18 (E124, Red 18; CI 16255), Acid Red 27 (E 123, CI 16185, C-Rot 46, Real Red D, FD&C Red Nr.2, Food Red 9, Naphthol red S), Acid Red 33 (Red 33, Fuchsia Red, D&C Red 33, CI 17200), Acid Red 35 (CI C.I.18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red n° 106 Pontacyl Brilliant Pink), Acid Red 73 (CI 27290), Acid Red 87 (Eosin, CI 45380), Acid Red 92 (COLIPA n° C.53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet n° 2, C.I. 60730, COLIPA n° C.063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent blue AE, Amido blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreenl), Acid Green 5 (CI 42095), Acid Green 9 (C.I.42100), Acid Green 22 (C.I.42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black n° 401, Naphthalene Black 10B, Amido Black 10B, CI 20 470, COLIPA n° B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

For example, the water solubility of anionic direct dyes can be determined in the following way. about 0.1 g of the anionic direct dye is placed in a beaker. A stir-fish is added. Then add about 100 ml of water. This mixture is heated to about 25° C. on a magnetic stirrer while stirring. It is stirred for about 60 minutes. The aqueous mixture is then visually assessed. If there are still undissolved residues, the amount of water is increased—for example in steps of about 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If about 0.1 g of the anionic direct dye dissolves in about 100 ml water at about 25° C., the solubility of the dye is about 1.0 g/L.

Acid Yellow 1 is called 8-hydroxy-5,7-dinitro-2-naphthalenesulfonic acid disodium salt and has a solubility in water of at least about 40 g/L (25° C.).

Acid Yellow 3 is a mixture of the sodium salts of mono- and sisulfonic acids of 2-(2-quinolyl)-1H-indene-1,3(2H)-dione and has a water solubility of about 20 g/L (25° C.).

Acid Yellow 9 is the disodium salt of 8-hydroxy-5,7-dinitro-2-naphthalenesulfonic acid, its solubility in water is above about 40 g/L (25° C.).

Acid Yellow 23 is the trisodium salt of4,5-dihydro-5-oxo-1-(4-sulfophenyl)-4-((4-sulfophenyl)azo)-1H-pyrazole-3-carboxylic acid and is highly soluble in water at 25° C.

Acid Orange 7 is the sodium salt of 4-[(2-hydroxy-1-naphthyl)azo]benzene sulphonate. Its water solubility is more than about 7 g/L (25° C.).

Acid Red 18 is the trinatirum salt of 7-hydroxy-8-[(E)-(4-sulfonato-1-naphthyl)-diazenyl)]-1,3-naphthalene disulfonate and has a very high-water solubility of more than about 20% by weight.

Acid Red 33 is the diantrium salt of 5-amino-4-hydroxy-3-(phenylazo)-naphthalene-2,7-disulphonate, its solubility in water is about 2.5 g/L (25° C.).

Acid Red 92 is the disodium salt of 3,4,5,6-tetrachloro-2-(1,4,5,8-tetrabromo-6-hydroxy-3-oxoxanthen-9-yl)benzoic acid, whose solubility in water is indicated as greater than about 10 g/L (25° C.).

Acid Blue 9 is the disodium salt of 2-({4-[N-ethyl(3-sulfonatobenzyl]amino]phenyl}{4-[(N-ethyl(3-sulfonatobenzyl)imino]-2,5-cyclohexadien-1-ylidene}methyl)-benzenesulfonate and has a solubility in water of more than about 20% by weight (25° C.).

Thermochromic dyes can also be used. Thermochromism involves the property of a material to change its color reversibly or irreversibly as a function of temperature. This can be done by changing both the intensity and/or the wavelength maximum.

Finally, it is also possible to use photochromic dyes. Photochromism involves the property of a material to change its color depending reversibly or irreversibly on irradiation with light, especially UV light. This can be done by changing both the intensity and/or the wavelength maximum.

Also, the preparation (B) may additionally contain one or more further ingredients selected from the group of solvents, thickening or film-forming polymers, surface-active compounds from the group of nonionic, cationic, anionic or zwitterionic/amphoteric surfactants, of the fatty components from the group of C8-C30 fatty alcohols, hydrocarbon compounds, fatty acid esters, acids and bases belonging to the group of pH regulators, perfumes, preservatives, plant extracts and protein hydrolysates.

Regarding the further preferred embodiments as contemplated herein and of the multicomponent packaging unit, mutatis mutandis what has been said about the process as contemplated herein applies.

### Examples

1. Preparation of the Silane Blends

### 1.1. Preparation of the Silane Blend (Silane Blend 1, Comparison)

A reactor with a heatable/coolable outer shell and with a capacity of 10 liters was filled with 4.67 kg of methyltrimethoxysilane. 1.33 kg of (3-aminopropyl)triethoxysilane was then added with stirring. This mixture was stirred at 30° C. Subsequently, 670 ml of water (dist.) was added dropwise with vigorous stirring, maintaining the temperature of the reaction mixture at 30° C. under external cooling. After completion of the water addition, stirring was continued for another 10 minutes. A vacuum of 700 mbar was then applied, and the reaction mixture was heated to a temperature of 44° C. Once the reaction mixture reached the temperature of 44° C., the ethanol and methanol released during the reaction were distilled off over a period of 40 minutes.

The distilled alcohols were collected in a chilled receiver. The reaction mixture was then allowed to cool to room temperature. To the mixture thus obtained, 3.33 kg of hexamethyldisiloxane was then dropped while stirring. It was stirred for 10 minutes. In each case, 100 ml of the silane blend was filled into a bottle with a capacity of 100 ml and screw cap closure with seal. After filling, the bottles were tightly closed.

### 1.2. Preparation of the Silane Blend (Silane Blend 2, Present Disclosure)

A reactor with a heatable/coolable outer shell and with a capacity of 10 liters was filled with 4.67 kg of methyltrimethoxysilane. 1.33 kg of (3-aminopropyl)triethoxysilane was then added with stirring. This mixture was stirred at 30° C. Subsequently, 670 ml of water (dist.) was added dropwise with vigorous stirring, maintaining the temperature of the reaction mixture at 30° C. under external cooling. After completion of the water addition, stirring was continued for another 10 minutes. A vacuum of 280 mbar was then applied, and the reaction mixture was heated to a temperature of 44° C. Once the reaction mixture reached the temperature of 44° C., the ethanol and methanol released during the reaction were distilled off over a period of 190 minutes.

During distillation, the vacuum was lowered to 200 mbar. The distilled alcohols were collected in a chilled receiver. The reaction mixture was then allowed to cool to room temperature. To the mixture thus obtained, 3.33 kg of hexamethyldisiloxane was then dropped while stirring. It was stirred for 10 minutes. In each case, 100 ml of the silane blend was filled into a bottle with a capacity of 100 ml and screw cap closure with seal. After filling, the bottles were tightly closed.

2. Measurement of the Content of C₁-C₆ Alcohols by GC-MS

Within 24 hours of their bottling, the ethanol and methanol contents of both silane blends were measured by GC-MS spectroscopy.

In a duplicate determination, one sample of each silane blend was analyzed by gas chromatography on a nonpolar column. Identification of the assigned components was performed by mass spectrometry using library comparison spectra (NIST/Wiley). Quantification was performed using internal standard calibration with methyl isobutyl ketone. The mean value was calculated from each duplicate determination.

| | | Silane |
|---|---|---|
| | Silane | blend 2 |
| | blend 1 | (present |
| | (comparison) | disclosure) |
| Methanol (% by weight) | 10.6/11.6 | 2.9/2.5 |
| Ethanol (% by weight) | 4.6/5.3 | 1.5/1.4 |
| Total C₁-C₆ alcohols | 16.1 | 4.2 |
| content (average) | | |

3. Coloring

The following colorant was provided (preparation (B)).

### Preparation (B)

| Colorona ® Bordeaux, Merck ® | 5.5 | g |
|---|---|---|
| MICA, CI 77491 (IRON OXIDES) | | |
| Hydroxyethyl cellulose | 1.0 | g |
| (Natrosol 250 HR) | | |
| PEG-12 Dimethicone | 2.0 | g |
| (Xiameter OFX-0193) | | |
| Water | Ad | |
| | 100 | g |

From each of the previously prepared bottles with silane blend, 20 g were weighed out (preparation A). The ready-to-use stain was prepared by shaking 20 g of preparation (A) and 100 g of preparation (B), respectively (shaking for 3 minutes). This mixture was then left to stand for 5 minutes.

For the application, one strand of hair (Kerling Euronaturhaar white) was dipped into the ready-to-use dye and left in it for 1 minute. After that, superfluous agent was stripped from each strand of hair. Then each strand of hair was washed with water and dried. Subsequently, the strands were visually evaluated under a daylight lamp. The following results were obtained:

| Silane blend 1 | Silane blend 2 |
|---|---|
| Comparison | Present disclosure |
| 20 g | 20 g |
| Colorant (B) | Colorant (B) |
| 100 g | 100 g |
| Color: burgundy red | Color: burgundy red |
| Color intensity: low | Color intensity: high |
| Hiding power: medium | Hiding power: medium |

1. A method for the preparation of an agent for the treatment of keratinous material, comprising the following steps:
(1) mixing one or more organic C₁-C₆ alkoxy silanes with water to produce a reaction mixture,
(2) removing a C₁-C₆ alcohol produced by a reaction between the C₁-C₆ alkoxy silanes with water in the reaction mixture, where the C₁-C₆ alcohol is partially or completely removed,
(3) optionally adding of one or more cosmetic ingredients to the reaction mixture, and
(4) filling a preparation produced by the reaction mixture into a packaging unit,
wherein the total content of C₁-C₆ alcohols in the preparation obtained in step (4)—based on a total weight of the preparation—is below about 10.0% by weight.

2. The method according to claim 1, wherein:
(1) mixing the one or more organic C₁-C₆ alkoxy silanes with the water comprises mixing an organic C₁-C₆ alcoxy silane of formula (I) and/or (II) with water,
R₁R₂N-L-Si(OR₃)ₐ(R₄)_{b} (I)
where
R₁, R₂ independently represent a hydrogen atom or a C₁-C₆ alkyl group,
L is a linear or branched divalent C₁-C₂₀ alkylene group,
R₁, R₄ independently of one another represent a C₁-C₆ alkyl group,
a, stands for an integer from 1 to 3, and
b stands for the integer 3-a, and
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
where
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III),
(A″″)-Si(R₆″)_{d}″(OR₅″)_{c}″ (III),
where
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g, and h is different from 0.

3. The method according to claim 1, wherein:
(1) mixing the one or more organic C₁-C₆ alkoxy silanes with the water comprises mixing an organic C₁-C₆ alkoxy silane of formula (IV) with water,
R₉Si(OR₁₀)ₖ(R₁₁)ₘ (IV),
where
R₉ represents a C₁-C₁₂ alkyl group,
R₁₀ represents a C₁-C₆ alkyl group,
R₁₁ represents a C₁-C₆ alkyl group
k is an integer from 1 to 3, and
m stands for the integer 3-k.

4. The method according to claim 1, wherein:
(1) mixing the one or more organic C₁-C₆ alkoxy silanes with the water comprises mixing the one or more organic C₁-C₆ alkoxy silanes with water in a reaction vessel.

5. The method according to claim 1, wherein:
(1) mixing the one or more organic C₁-C₆ alkoxy silanes with the water comprises mixing the one or more organic C₁-C₆ alkoxy silanes with from about 0.10 to about 0.80 molar equivalents of water (S-W),
where the molar equivalents of water are calculated according to the formula
S−W=(mol(water)/(mol(silane)×n(alkoxy))
with
S-W=molar equivalent water
mol(water)=molar quantity of water used
mol(silanes)=total molar amount of C₁-C₆ alkoxy silanes used in the reaction
n(alkoxy)=number of C₁-C₆ alkoxy groups per C₁-C₆ alkoxy silane

6. The method according to claim 1, further comprising:
(1) reactiong the one or more organic C₁-C₆ alkoxy silanes with water at a temperature of from about 20 to about 65° C.

7. The method according to claim 1, wherein:
(2) removing the C₁-C₆ alcohols produced by the reaction in step (1) from the reaction mixture comprising removing the C₁-C₆ alcohols at a temperature of from about 20 to about 65° C.

8. The method according to claim 1, wherein:
(2) removing the C₁-C₆ alcohols produced by the reaction in step (1) from the reaction mixture comprises distilling the reaction mixture at a pressure of from 0 to about 900 mbar.

9. The method according to claim 1, wherein:
(2) removing the C₁-C₆ alcohols produced by the reaction in step (1) from the reaction mixture comprises distilling the reaction mixture over a period of from about 90 to about 300 minutes.

10. The method according to claim 1, wherein:
the one or more cosmetic ingredients are selected from the group of polymers, surface-active compounds, coloring compounds, lipid components, pH regulators, perfumes, preservatives, plant extracts, protein hydrolysates, and combinations thereof.

11. The method according to claim 1, wherein:
the one or more cosmetic ingredients are selected from the group of hexamethyldisiloxane, octamethyltrisiloxane, decamethyltetrasiloxane, hexamethylcyclotrisiloxane, octamethylcyclotetrasiloxane and/or decamethylcyclopentasiloxane.

12. The method according to claim 1, wherein:
(4) Filling the preparation produced by the reaction mixture comprises filling the preparation into a bottle, tube, jar, can, sachet, aerosol pressure container, non-aerosol pressure container, canister, hobbock, or combination thereof.

13. The method according to claim 1, wherein a total content of the C₁-C₆ alcohols in the preparation obtained after step (4)—based on a total weight of the preparation—is below about 9.0% by weight.

14. The method according to claim 1, wherein a water content in the preparation obtained after step (4)—based on a total weight of the preparation—is below about 5.0% by weight.

15. The method of claim 1, comprising the steps in the following order:
(1) mixing the one or more organic C₁-C₆ alkoxy silanes with the water,
(2) removing the C₁-C₆ alcohols produced in step (1),
(3) adding the one or more cosmetic ingredients, and
(4) filling of the preparation into the packaging unit.

16. The method of claim 1, comprising the steps in the following order:
(1) reacting the one or more organic C₁-C₆ alkoxy silanes with the water,
(3) adding the one or more cosmetic ingredients,
(2) removing the C₁-C₆ alcohols produced in step (1), and
(4) filling the preparation into the packaging unit.

17. The method according to claim 1, wherein the preparation comprises an agent for coloring keratinous material, for maintaining keratinous material, or for changing the shape of keratinous material.

18. An agent for treating keratinous material comprising a preparation in a packaging unit prepared by the method described in claim 1.

19. A multicomponent packaging unit (kit-of-parts) for dyeing keratinous material, comprising:
a first packaging unit containing a cosmetic preparation (A) and
a second packaging unit containing a cosmetic preparation (B),
where
the cosmetic preparation (A) in the first packaging unit has been prepared by the method described in claim 1, and
the cosmetic formulation (B) comprises at least one colorant compound selected from the group of pigments, direct dyes and/or oxidation dye precursors.

20. The method according to claim 1, wherein a total content of the C₁-C₆ alcohols in the preparation obtained after step (4)—based on a total weight of the preparation—is below about 5.0% by weight
